(19) United States
(12) Patent Application Publication
(10) Pub. No.: US 2023/0235190 A1
(43) Pub. Date: Jul. 27, 2023

(54) FLUORESCENT COMPOSITION COMPRISING AT LEAST ONE BENZAZOLE COMPOUND FOR THE SECUREMENT OF PRODUCTS

(71) Applicant: CRIME SCIENCE TECHNOLOGY, Lille (FR)

(72) Inventors: Cosimo PRETE, VILLENEUVE D’ASCQ (FR); Jérémy MALINGE, ANGERS (FR); Alexis DEPAUW, GIF SUR YVETTE (FR)

(73) Assignee: CRIME SCIENCE TECHNOLOGY, Lille (FR)

(21) Appl. No.: 18/009,171

(22) PCT Filed: Jun. 10, 2021
(86) PCT No.: PCT/FR2021/051044

§ 371 Date: Dec. 8, 2022

(30) Foreign Application Priority Data
Jun. 10, 2020 (FR) 20 06079

(51) Int. Cl. C09D 11/50; C07D 277/66; C09K 11/06; C09K 11/02; C09B 57/00; C09B 67/20; C09B 67/22; C09D 11/328; C09D 11/037; B42D 25/387; B41M 3/06

(52) U.S. Cl. CPC C09D 11/50; B41M 3/06; B42D 25/387; C07D 277/66; C09B 57/00; C09B 67/0033; C09B 67/0063; C09D 11/037; C09D 11/328; C09K 11/02; C09K 11/06; B42D 25/23; C09K 2211/1007; C09K 2211/1014; C09K 2211/1022

(57) ABSTRACT

A fluorescent composition for the securement of a product, and the method for the securement of a product, which includes the step of preparing the fluorescent composition and a securement step by applying the fluorescent composition to at least a portion of the product. The fluorescent composition includes a polymer matrix incorporating at least a compound having formula I:

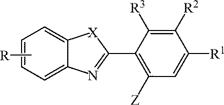

### TECHNICAL FIELD

The present invention relates to the field of product securement and authentication. More specifically, the invention relates to a fluorescent composition for securing and authenticating products such as identity, fiduciary and administrative documents.

### PRIOR ART

Counterfeiting and forgery are growing significantly in many sectors such as that of packaging, particularly medicinal product blister packaging, but also in high-added value sectors such as the luxury, automotive or aeronautics sectors. With the growth of identity theft and introduction particularly of points-based driving licences in some countries, identity and administrative documents are also the target of forgeries. Therefore, the securement and authentication of products is essential and involves national and international security challenges.

In relation to the securement of products such as an identity, fiduciary or administrative document, various companies provide visual authentication solutions, for example using holograms or laser etching, which can be used to insert information on a plastic card body such as an identify card, a health card or a driving licence.

The document EP0708935 A1 describes for example a set of holographic protection layers. This set consists of a backing film having at least one layer formed by a protective varnish, a reflective or transparent layer bearing a diffracting microstructure, and finally, an adhesive layer. Once the set of layers has been transferred to a document, the securement thereof is obtained. As described in the document WO2010/086522, this system was subsequently enhanced by means of perforations for rendering the separation of the various layers more difficult. However, the set of layers, even perforated, is formed from a multitude of parts that need to be assembled, which represents additional constraints in terms of time and cost.

The document FR 16 50164, of which the Applicant is the holder, describes the use of a compound of the 4,4-difluoro-4-bora-3a,4a-diaza-s-indacene family for the preparation of a securement element of a product, particularly a document, said securement element comprising a polymer and the compound being incorporated in said polymer. Although interesting, the use of the compounds described only makes it possible to obtain fluorescence emissions within the range from 500 nm to infrared, and thus does not cover the entire visible spectrum range.

The application US 2008/0081913 A1 describes benzoxazole and benzothiazole type compounds having fluorescent properties and being useful particularly as authentication compounds in data storage media and data storage substrates.

The products, and particularly documents, can be secured by security elements that can be classified according to three security levels based on the means used for the detection thereof. Thus, level 1 security elements are elements that can be detected by at least one of the five senses or by means of a contrasting background. This level will particularly include guilloche patterns, optical variability devices such as iridescent printing, holograms, optically variable inks, taggants, changeable laser images, or multiple laser images.

Level 2 security elements are elements detectable by means of simple equipment such as an ultraviolet lamp, a convex lens, or a mobile phone flashlight. This level will include detectable elements such as microprints, fluorescent inks, and fluorescent fibres or chips.

Finally, level 3 security elements are elements detectable by means of complex equipment such as for example a spectrofluorometer or an electron microscope. This category particularly includes nano-etched pigments, biometric chips and fluorescent taggants not detectable to the naked eye.

As a general rule, a secured product incorporates several security elements of different levels.

Although existing securement solutions prove to be interesting, some may be difficult to implement and/or check and the need to develop alternatives, particularly in the visible spectrum, is still present.

Therefore, there is a real need to develop novel securement means that are easy to implement, stable, having fluorescence emissions and enabling a rapid check of the authenticity of the products. These novel means must provide a high level of security and must not be exclusive from each other and from already existing means.

Therefore, it is to the inventors’ credit that they have achieved all or some of these aims by identifying and developing compounds which make it possible to obtain a particularly advantageous fluorescent composition for use in the field of product securement.

### SUMMARY

The present invention relates to a fluorescent composition comprising a polymer matrix incorporating a compound having formula I:

### [Chem. 1]

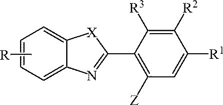

wherein,
X is chosen from NH, O and S;
Z is chosen from OH, NHR⁵ and N(R⁵)2;
R is chosen from hydrogen, C1-C6-alkyl, C3-C6-cycloalkyl, aryl, vinyl, ethynyl, halogen, —NO₂, —NH₂, —NHR⁴, —N(R⁴)₂, —N⁺(R⁴)₃, —NHCOR⁴, —CHO, —C(O)OH, —C(O)OR⁴, —CF₃, C1-C6-alkoxy, aryloxy, —SH, —SO₃H, —SR⁴;
R¹, R² and R³ are independently chosen from hydrogen, C1-C6-alkyl, C3-C6-cycloalkyl, aryl, vinyl, ethynyl, halogen, —NO₂, —NH₂, —NHR⁴, —N(R⁴)₂, —N⁺(R⁴)₃, —NHCOR⁴, —CHO, —C(O)OH, —C(O)OR⁴, —CF₃, C1-C6-alkoxy, aryloxy, —SH, —SO₃H, —SR⁴;
R⁴ is chosen from C1-C6-alkyl, C3-C6-cycloalkyl and aryl;
R⁵ is chosen from C1-C6-alkyl, hydroxy-C1-C6-alkyl, halogen, aryl, acyl, C1-C6-alkyloxycarbonyl, C1-C6-alkylaminocarbonyl, C1-C6-alkylaminothionyl, di-C1-C6-alkylaminothionyl, arylaminocarbonyl, arylaminothionyl, arylsulfonyl, C1-C6-alkylsulfonyl, cinnamoyl, benzoyl, 2,3,4,5,6-pentahalogenobenzoyl and 2,3,5,6-tetrahalogenobenzoyl, optionally substituted by C1-C6-alkyl, C1-C6-alkoxy, trifluoro-C1-C6-alkyl, C1-C6-alkylamino or hydroxy-C1-C6-alkylamino; with the condition that Z is not OH when R² is —NHCOR⁴; and wherein the symbol

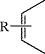

means that R can be substituted several times at any free position of the benzene nucleus.

According to a further aspect, the use of a fluorescent composition according to the invention for the securement of a product is proposed.

According to a further aspect, a method is proposed for the securement of a product comprising a step of preparing a fluorescent composition as defined above and a step of securement by applying said fluorescent composition on at least a portion of the product to be secured.

According to a further aspect, a compound is proposed having formula (II):

### [Chem. 2.]

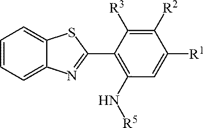

wherein,
R¹, and R² and R³ are independently chosen from hydrogen, C1-C6-alkyl, C3-C6-cycloalkyl, aryl, vinyl, ethynyl, halogen, —NO₂, —NH₂, —NHR⁴, —N(R⁴)₂, —N⁺(R⁴)₃, —NHCOR⁴, —CHO, —C(O)OH, —C(O)OR⁴, —CF₃, C1-C6-alkoxy, aryloxy, —SH, —SO₃H, —SR⁴;
R⁴ is chosen from C1-C6-alkyl, C1-C6-cycloalkyl and aryl;
R⁵ is chosen from C1-C6-alkyl, hydroxy-C1-C6-alkyl, C1-C6-alkyloxycarbonyl, di-C1-C6-alkylaminothionyl, C1-C6-alkylsulfonyl, cinnamoyl, benzoyl, 2,3,4,5,6-pentahalogenobenzoyl and 2,3,5,6-tetrahalogenobenzoyl, optionally substituted by C1-C6-alkyl, C1-C6-alkoxy, trifluoro-C1-C6-alkyl, C1-C6-alkylamino or hydroxy-C1-C6-alkylamino; and
with the condition that:
R¹, R² and R³ are not all three hydrogen when R⁵ is cinnamoyl, or benzoyl optionally substituted by methyl, methoxy, chloro or trifluoromethyl;
R¹ is not methyl when R⁵ is benzoyl;
R² is not methyl when R⁵ is benzoyl; and
R³ is not methyl, methoxy or chloro when R⁵ is benzoyl.

### DETAILED DESCRIPTION OF THE INVENTION

The present invention firstly relates to a fluorescent composition comprising a polymer matrix incorporating a compound having formula I:

### [Chem. 1]

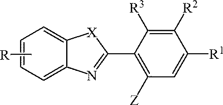

wherein,
X is chosen from NH, O and S; preferably X is S;
Z is chosen from OH, NHR⁵ and N(R⁵)₂; preferably Z is NHR⁵ or N(R⁵)₂; more preferably Z is NHR⁵;
R is chosen from hydrogen, C1-C6-alkyl, C3-C6-cycloalkyl, aryl, vinyl, ethynyl, halogen, —NO₂, —NH₂, —NHR⁴, —N(R⁴)₂, —N⁺(R⁴)₃, —NHCOR⁴, —CHO, —C(O)OH, —C(O)OR⁴, —CF₃, aryloxy, —SH, —SO₃H, —SR⁴; preferably R is chosen from hydrogen, C1-C4-alkyl, halogen, —NO₂, —NH₂, —NHR⁴, —N(R⁴)₂, C1-C4-alkoxy; more preferably R is chosen from hydrogen, C1-C2-alkyl, halogen, C1-C4-alkoxy; more preferably, R is hydrogen;
R¹, R² and R³ are independently chosen from hydrogen, C1-C6-alkyl, C3-C6-cycloalkyl, aryl, vinyl, ethynyl, halogen, —NO₂, —NH₂, —NHR⁴, —N(R⁴)₂, —N⁺(R⁴)₃, —NHCOR⁴, —CHO, —C(O)OH, —C(O)OR⁴, —CF₃, C1-C6-alkoxy, aryloxy, —SH, —SO₃H, —SR⁴; preferably R¹, R² and R³ are independently chosen from hydrogen, C1-C6-alkyl, C1-C6-alkoxy, halogen and —NO₂; more preferably R¹, R² and R³ are independently chosen from hydrogen, C1-C4-alkyl, C1-C4-alkoxy, halogen and —NO₂; more preferably R¹, R² and R³ are independently chosen from hydrogen, C1-C2-alkyl, C1-C2-alkoxy, halogen and —NO₂; even more preferably R¹, R² and R³ are independently chosen from hydrogen, methyl, methoxy, bromo, chloro and —NO₂;
R⁴ is chosen from alkyl, cycloalkyl and aryl; preferably R⁴ is chosen from C1-C6-alkyl, C3-C6-cycloalkyl and aryl;
R⁵ is chosen from C1-C6-alkyl, hydroxy-C1-C6-alkyl, halogen, aryl, acyl, C1-C6-alkyloxycarbonyl, C1-C6-alkylaminocarbonyl, C1-C6-alkylaminothionyl, di-C1-C6-alkylaminothionyl, arylaminocarbonyl, arylaminothionyl, arylsulfonyl, C1-C6-alkylsulfonyl, cinnamoyl, benzoyl, 2,3,4,5,6-pentahalogenobenzoyl and 2,3,5,6-tetrahalogenobenzoyl, optionally substituted by C1-C6-alkyl, C1-C6-alkoxy, trifluoro-C1-C6-alkyl, C1-C6-alkylamino or hydroxy-C1-C6-alkylamino; preferably R⁵ is chosen from cinnamoyl, benzoyl, 2,3,4,5,6-pentahalogenobenzoyl and 2,3,5,6-tetrahalogenobenzoyl, optionally substituted by C1-C6-alkyl, C1-C6-alkoxy, trifluoro-C1-C6-alkyl, C1-C6-alkylamino or hydroxy-C1-C6-alkylamino; more preferably R⁵ is chosen from cinnamoyl, benzoyl, 2,3,4,5,6-pentahalogenobenzoyl and 2,3,5,6-tetrafluorobenzoyl, optionally substituted by C1-C4-alkyl, C1-C4-alkoxy, trifluoro-C1-C4-alkyl, C1-C4-alkylamino or hydroxy-C1-C4-alkylamino; more preferably R⁵ is chosen from cinnamoyl, benzoyl, 2,3,4,5,6-pentahalogenobenzoyl and 2,3,5,6-tetrafluorobenzoyl, optionally substituted by methyl, methoxy, trifluoromethyl, butylamino or hydroxyethylamino; for example R⁵ is chosen from benzoyl, 4-(trifluoromethyl)benzoyl, 3,5-bis(trifluoromethyl)benzoyl, 2,3,4,5,6-pentafluorobenzoyl, 4-(butylamino)-2,3,5,6-tetrafluorobenzoyl, cinnamoyl and 4-((2-hydroxyethyl)amino)benzoyl; in particular R⁵ is chosen from benzoyl, 3,5-bis(trifluoromethyl)benzoyl, 4-(butylamino)-2,3,5,6-tetrafluorobenzoyl and cinnamoyl with the condition that Z is not OH when R¹ is —NHCOR⁴.

It is understood in the present application that when several preference levels are given for different substituents of a Markush formula, different preference levels can be combined with each other. In other words, it is understood that all combinations of the different preference levels are explicitly envisaged.

The polymer matrix of the fluorescent composition according to the invention can be obtained from an amorphous or semi-crystalline polymer chosen from polycarbonate, polystyrene, polyethylene, polypropylene, polyethylene terephthalate, polyacrylate, polymethacrylate, polyvinyl chloride, polyamides, polyaramids, ethylene-vinyl acetate, polyurethane, thermoplastic polyurethane, cyanoacrylate, rosin resins, pine resins, photopolymerisable resins, acrylic and mixtures thereof. Preferably, the polymer matrix of the fluorescent composition can be obtained from a polymer chosen from polycarbonate, polyethylene, thermoplastic polyurethane, acrylic and photopolymerisable resins and mixtures thereof, and more preferably the polymer matrix is a polycarbonate or polypropylene matrix. For example, the polymer matrix of the fluorescent composition according to the invention can be obtained from a polymer chosen from polycarbonate, polystyrene, polyethylene, polypropylene, polyethylene terephthalate, polyacrylate, polymethacrylate, polyvinyl chloride, polyamides, polyaramids, polyurethane, thermoplastic polyurethane (TPU), cyanoacrylate, rosin resins, pine resins, photopolymerisable resins, acrylic and mixtures thereof. In particular, the polymer matrix of the fluorescent composition can be obtained from a polymer chosen from polycarbonate, polyethylene, polypropylene, polyethylene terephthalate, thermoplastic polyurethane, photopolymerisable resins, acrylic, and mixtures thereof. More specifically, the polymer matrix of the fluorescent composition can be obtained from a polymer chosen from polycarbonate, polyethylene, polypropylene, polyethylene terephthalate and mixtures thereof. Even more specifically, the polymer matrix of the fluorescent composition can be obtained from a polymer chosen from polycarbonate, polypropylene, polyethylene terephthalate and mixtures thereof. More specifically again, the polymer matrix of the fluorescent composition can be obtained from polycarbonate or polyethylene terephthalate.

According to a specific embodiment, the polymer matrix is a semicrystalline polymer matrix.

Advantageously, the polymer matrix does not contain anti-UV additives in order to thus enable an optimal maintenance of the fluorescence properties. Similarly, it is also advantageous to use a polymer matrix which retains the transparency properties thereof even after a shaping step.

The expression “polymer matrix incorporating a compound having formula I” means according to the present invention that the compound having formula I is intimately integrated in the polymer matrix so as to form a mixture. Preferably, the compound having formula I is intimately integrated in the polymer matrix so as to form a homogeneous mixture having no dispersion. The integration of the compound in the polymer matrix can for example be carried out hot. In this scenario, the polymer matrix is heated to the melting point thereof, then the compound having formula I is added into the molten mass before the whole is mixed. Thus, the compound can be integrated in the polymer matrix by molten process, extrusion, calendering extrusion, spinning extrusion, plastic injection or dyeing.

Particularly advantageously, the inventors observed that the compounds having formula I according to the invention could be incorporated in polymer matrixes without altering the performances of the matrix, or above all those of the incorporated compounds.

In an embodiment, the compounds incorporated in the polymer matrix of the fluorescent composition are the compounds having formula I wherein X is S.

Thus, according to this embodiment, the compounds incorporated in the polymer matrix of the fluorescent composition are those having formula Ia:

### [Chem. 3.]

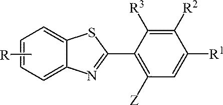

wherein R, R¹, R², R³ and Z are as defined in formula I.

Preferred compounds having formula Ia are those wherein R, R¹, R², R³ and/or Z are defined as follows:
Z is NHR⁵ or N(R⁵)₂;
R is chosen from hydrogen, C1-C4-alkyl, halogen, —NO₂ and C1-C4-alkoxy; preferably R is hydrogen;
R¹, R² and R³ are independently chosen from hydrogen, C1-C6-alkyl, C1-C6-alkoxy, halogen and —NO₂; preferably R¹, R² and R³ are independently chosen from hydrogen, C1-C4-alkyl, C1-C4-alkoxy, halogen and —NO₂; more preferably R¹, R² and R³ are independently chosen from hydrogen, C1-C2-alkyl, C1-C2-alkoxy, Cl, F and —NO₂; even more preferably R¹, R² and R³ are independently chosen from hydrogen, methyl, methoxy, Cl, F and —NO₂;
R⁵ is chosen from C1-C6-alkyl, hydroxy-C1-C6-alkyl, halogen, aryl, acyl, C1-C6-alkyloxycarbonyl, di-C1-C6-alkylaminothionyl, arylaminocarbonyl, arylsulfonyl, C1-C6-alkylsulfonyl, cinnamoyl, benzoyl and 2,3,5,6-tetrahalogenobenzoyl, optionally substituted by C1-C6-alkyl, C1-C6-alkoxy, trifluoro-C1-C6-alkyl, C1-C6-alkylamino or hydroxy-C1-C6-alkylamino; preferably R⁵ is chosen from cinnamoyl, benzoyl and 2,3,5,6-tetrahalogenobenzoyl, optionally substituted by C1-C6-alkyl, C1-C6-alkoxy, trifluoro-C1-C6-alkyl, C1-C6-alkylamino or hydroxy-C1-C6-alkylamino; more preferably R⁵ is chosen from cinnamoyl, benzoyl and 2,3,5,6-tetrafluorobenzoyl, optionally substituted by C1-C4-alkyl, C1-C4-alkoxy, trifluoro-C1-C4-alkyl, C1-C4-alkylamino or hydroxy-C1-C4-alkylamino; more preferably R⁵ is chosen from cinnamoyl, benzoyl and 2,3,5,6-tetrafluorobenzoyl, optionally substituted by methyl, methoxy, trifluoromethyl, butylamino or hydroxyethylamino; for example R⁵ is chosen from benzoyl, 4-(trifluoromethyl)benzoyl, 3,5-bis(trifluoromethyl)benzoyl, 2,3,4,5,6-pentafluorobenzoyl, 4-(butylamino)-2,3,5,6-tetrafluorobenzoyl, cinnamoyl and 4-((2-hydroxyethyl)amino)benzoyl; in particular R⁵ is chosen from benzoyl, 3,5-bis(trifluoromethyl)benzoyl, 4-(butylamino)-2,3,5,6-tetrafluorobenzoyl and cinnamoyl.

According to an alternative embodiment of this embodiment, the compounds incorporated in the polymer matrix of the fluorescent composition are those having formula Ib:

### [Chem. 4]

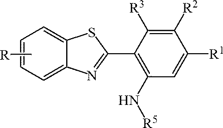

wherein R, R¹, R², R³ and R⁵ are as defined in formula I.

Preferred compounds having formula Ib are those wherein R, R¹, R², R³ and R⁵ are defined as follows:
R is chosen from hydrogen, C1-C4-alkyl, halogen, —NO₂ and C1-C4-alkoxy; preferably R is hydrogen;
R¹, R² and R³ are independently chosen from hydrogen, C1-C6-alkyl, C1-C6-alkoxy, halogen and —NO₂; preferably R¹, R² and R³ are independently chosen from hydrogen, C1-C4-alkyl, C1-C4-alkoxy, halogen and —NO₂; more preferably R¹, R² and R³ are independently chosen from hydrogen, C1-C2-alkyl, C1-C2-alkoxy, Cl, F and —NO₂; even more preferably R¹, R² and R³ are independently chosen from hydrogen, methyl, methoxy, Cl, F and —NO₂;
R⁵ is chosen from cinnamoyl, benzoyl and 2,3,5,6-tetrahalogenobenzoyl, optionally substituted by C1-C6-alkyl, C1-C6-alkoxy, trifluoro-C1-C6-alkyl, C1-C6-alkylamino or hydroxy-C1-C6-alkylamino; preferably R⁵ is chosen from cinnamoyl, benzoyl and 2,3,5,6-tetrafluorobenzoyl, optionally substituted by C1-C4-alkyl, C1-C4-alkoxy, trifluoro-C1-C4-alkyl, C1-C4-alkylamino or hydroxy-C1-C4-alkylamino; more preferably R⁵ is chosen from cinnamoyl, benzoyl and 2,3,5,6-tetrafluorobenzoyl, optionally substituted by methyl, methoxy, trifluoromethyl, butylamino or hydroxyethylamino; for example R⁵ is chosen from benzoyl, 4-(trifluoromethyl)benzoyl, 3,5-bis(trifluoromethyl)benzoyl, 2,3,4,5,6-pentafluorobenzoyl, 4-(butylamino)-2,3,5,6-tetrafluorobenzoyl, cinnamoyl and 4-((2-hydroxyethyl)amino)benzoyl; in particular R⁵ is chosen from benzoyl, 3,5-bis(trifluoromethyl)benzoyl, 4-(butylamino)-2,3,5,6-tetrafluorobenzoyl and cinnamoyl.

According to a further alternative embodiment of this embodiment, the compounds incorporated in the polymer matrix of the fluorescent composition are those having formula Ic:

### [Chem. 5]

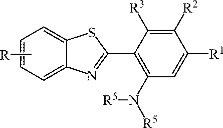

wherein R, R¹, R², R³ and R⁵ are as defined in formula I.

In an embodiment, the compounds incorporated in the polymer matrix of the fluorescent composition are the compounds having formula I wherein Z is NHR⁵ or N(R⁵)₂, preferably Z is NHR⁵.

In an embodiment, the compounds incorporated in the polymer matrix of the fluorescent composition are the compounds having formula I wherein R is hydrogen.

Particularly preferred compounds having formula I incorporated in the polymer matrix of the invention are those listed in Table 1 hereinafter:

TABLE 1

| Compound | Structure | Name |
|---|---|---|
| 1 | 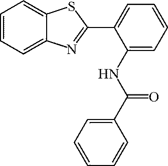 [SMILES: O=C(Nc1ccccc1-c1nc2ccccc2s1)c1ccccc1] | N-(2-(benzo[d]thiazol-2-yl)phenyl)benzamide |
| 2 | 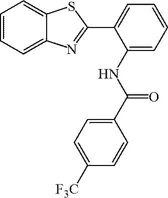 [SMILES: O=C(Nc1ccccc1-c1nc2ccccc2s1)c1ccc(C(F)(F)F)cc1] | N-(2-(benzo[d]thiazol-2-yl)phenyl)-4-(trifluoromethyl)benzamide |
| 3 | 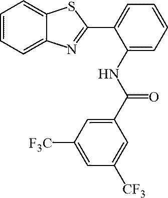 [SMILES: O=C(Nc1ccccc1-c1nc2ccccc2s1)c1cc(C(F)(F)F)cc(C(F)(F)F)c1] | N-(2-(benzo[d]thiazol-2-yl)phenyl)-3,5-bis(trifluoromethyl)benzamide |
| 4 | 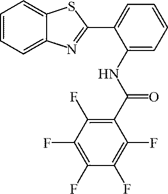 [SMILES: O=C(Nc1ccccc1-c1nc2ccccc2s1)c1c(F)c(F)c(F)c(F)c1F] | N-(2-(benzo[d]thiazol-2-yl)phenyl)-2,3,4,5,6-pentafluorobenzamide |
| 5 | 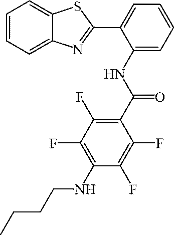 [SMILES: CCCCNc1c(F)c(F)c(C(=O)Nc2ccccc2-c2nc3ccccc3s2)c(F)c1F] | N-(2-(benzo[d]thiazol-2-yl)phenyl)-4-(butylamino)-2,3,5,6-tetrafluorobenzamide |
| 6 | 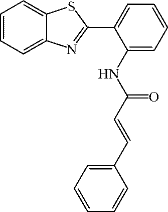 [SMILES: O=C(/C=C/c1ccccc1)Nc1ccccc1-c1nc2ccccc2s1] | N-(2-(benzo[d]thiazol-2-yl)phenyl)cinnamamide |
| 7 | 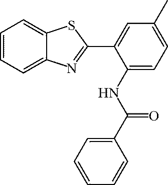 [SMILES: Cc1ccc(NC(=O)c2ccccc2)c(-c2nc3ccccc3s2)c1] | N-(2-(benzo[d]thiazol-2-yl)-4-methylphenyl)benzamide |
| 8 | 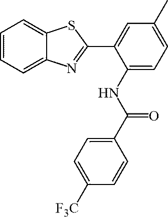 [SMILES: Cc1ccc(NC(=O)c2ccc(C(F)(F)F)cc2)c(-c2nc3ccccc3s2)c1] | N-(2-(benzo[d]thiazol-2-yl)-4-methylphenyl)-4-(trifluoromethyl)benzamide |
| 9 | 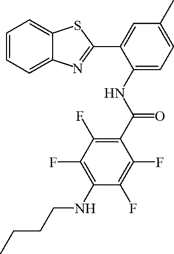 [SMILES: CCCCNc1c(F)c(F)c(C(=O)Nc2ccc(C)cc2-c2nc3ccccc3s2)c(F)c1F] | N-(2-(benzo[d]thiazol-2-yl)-4-methylphenyl)-4-(butylamino)-2,3,5,6-tetrafluorobenzamide |
| 10 | 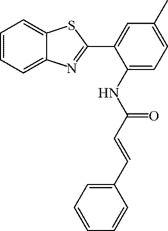 [SMILES: Cc1ccc(NC(=O)/C=C/c2ccccc2)c(-c2nc3ccccc3s2)c1] | N-(2-(benzo[d]thiazol-2-yl)-4-methylphenyl)cinnamamide |
| 11 | 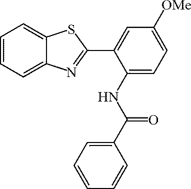 [SMILES: COc1ccc(NC(=O)c2ccccc2)c(-c2nc3ccccc3s2)c1] | N-(2-(benzo[d]thiazol-2-yl)-4-methoxyphenyl)benzamide |
| 12 | 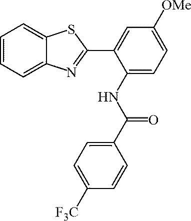 [SMILES: COc1ccc(NC(=O)c2ccc(C(F)(F)F)cc2)c(-c2nc3ccccc3s2)c1] | N-(2-(benzo[d]thiazol-2-yl)-4-methoxyphenyl)-4-(trifluoromethyl)benzamide |
| 13 | 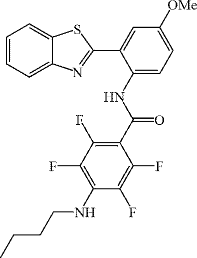 [SMILES: CCCCNc1c(F)c(F)c(C(=O)Nc2ccc(OC)cc2-c2nc3ccccc3s2)c(F)c1F] | N-(2-(benzo[d]thiazol-2-yl)-4-methoxyphenyl)-4-(butylamino)-2,3,5,6-tetrafluorobenzamide |
| 14 | 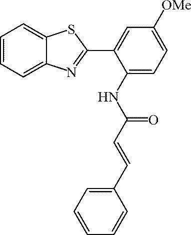 [SMILES: COc1ccc(NC(=O)/C=C/c2ccccc2)c(-c2nc3ccccc3s2)c1] | N-(2-(benzo[d]thiazol-2-yl)-4-methoxyphenyl)cinnamamide |
| 15 | 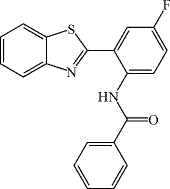 [SMILES: O=C(Nc1ccc(F)cc1-c1nc2ccccc2s1)c1ccccc1] | N-(2-(benzo[d]thiazol-2-yl)-4-fluorophenyl)benzamide |
| 16 | 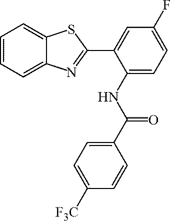 [SMILES: O=C(Nc1ccc(F)cc1-c1nc2ccccc2s1)c1ccc(C(F)(F)F)cc1] | N-(2-(benzo[d]thiazol-2-yl)-4-fluorophenyl)-4-(trifluoromethyl)benzamide |
| 17 | 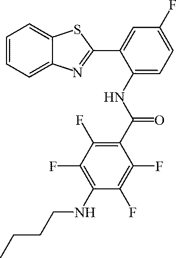 [SMILES: CCCCNc1c(F)c(F)c(C(=O)Nc2ccc(F)cc2-c2nc3ccccc3s2)c(F)c1F] | N-(2-(benzo[d]thiazol-2-yl)-4-fluorophenyl)-4-(butylamino)-2,3,5,6-tetrafluorobenzamide |
| 18 | 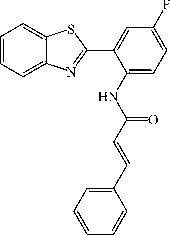 [SMILES: O=C(/C=C/c1ccccc1)Nc1ccc(F)cc1-c1nc2ccccc2s1] | N-(2-(benzo[d]thiazol-2-yl)-4-fluorophenyl)cinnamamide |
| 19 | 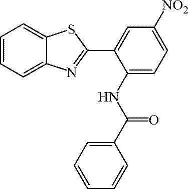 [SMILES: O=C(Nc1ccc([N+](=O)[O-])cc1-c1nc2ccccc2s1)c1ccccc1] | N-(2-(benzo[d]thiazol-2-yl)-4-nitrophenyl)benzamide |
| 20 | 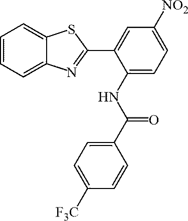 [SMILES: O=C(Nc1ccc([N+](=O)[O-])cc1-c1nc2ccccc2s1)c1ccc(C(F)(F)F)cc1] | N-(2-(benzo[d]thiazol-2-yl)-4-nitrophenyl)-4-(trifluoromethyl)benzamide |
| 21 | 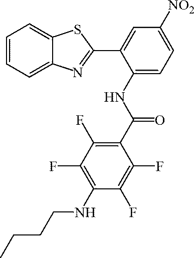 [SMILES: CCCCNc1c(F)c(F)c(C(=O)Nc2ccc([N+](=O)[O-])cc2-c2nc3ccccc3s2)c(F)c1F] | N-(2-(benzo[d]thiazol-2-yl)-4-nitrophenyl)-4-(butylamino)-2,3,5,6-tetrafluorobenzamide |
| 22 | 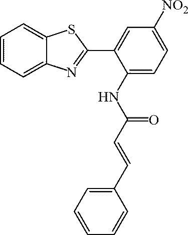 [SMILES: O=C(/C=C/c1ccccc1)Nc1ccc([N+](=O)[O-])cc1-c1nc2ccccc2s1] | N-(2-(benzo[d]thiazol-2-yl)-4-nitrophenyl)cinnamamide |
| 23 | 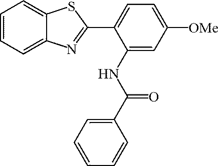 [SMILES: COc1ccc(-c2nc3ccccc3s2)c(NC(=O)c2ccccc2)c1] | N-(2-(benzo[d]thiazol-2-yl)-5-methoxyphenyl)benzamide |
| 24 | 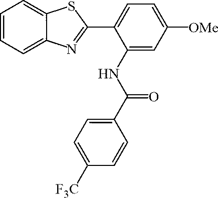 [SMILES: COc1ccc(-c2nc3ccccc3s2)c(NC(=O)c2ccc(C(F)(F)F)cc2)c1] | N-(2-(benzo[d]thiazol-2-yl)-5-methoxyphenyl)-4-(trifluoromethyl)benzamide |
| 25 | 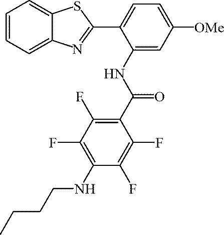 [SMILES: CCCCNc1c(F)c(F)c(C(=O)Nc2cc(OC)ccc2-c2nc3ccccc3s2)c(F)c1F] | N-(2-(benzo[d]thiazol-2-yl)-5-methoxyphenyl)-4-(butylamino)-2,3,5,6-tetrafluorobenzamide |
| 26 | 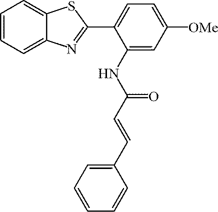 [SMILES: COc1ccc(-c2nc3ccccc3s2)c(NC(=O)/C=C/c2ccccc2)c1] | N-(2-(benzo[d]thiazol-2-yl)-5-methoxyphenyl)cinnamamide |
| 27 | 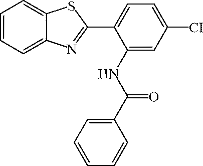 | N-(2-(benzo[d]thiazol-2-yl)-5-chlorophenyl)benzamide |
| 28 | 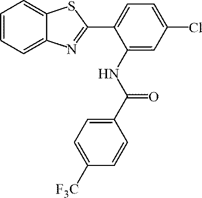 [SMILES: O=C(Nc1cc(Cl)ccc1-c1nc2ccccc2s1)c1ccc(C(F)(F)F)cc1] | N-(2-(benzo[d]thiazol-2-yl)-5-chlorophenyl)-4-(trifluoromethyl)benzamide |
| 29 | 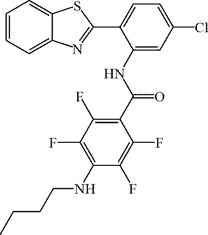 [SMILES: CCCCNc1c(F)c(F)c(C(=O)Nc2cc(Cl)ccc2-c2nc3ccccc3s2)c(F)c1F] | N-(2-(benzo[d]thiazol-2-yl)-5-chlorophenyl)-4-(butylamino)-2,3,5,6-tetrafluorobenzamide |
| 30 | 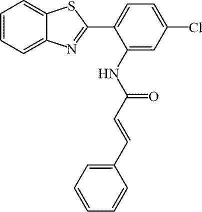 [SMILES: O=C(/C=C/c1ccccc1)Nc1cc(Cl)ccc1-c1nc2ccccc2s1] | N-(2-(benzo[d]thiazol-2-yl)-5-chlorophenyl)cinnamamide |
| 31 | 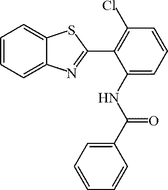 [SMILES: O=C(Nc1cccc(Cl)c1-c1nc2ccccc2s1)c1ccccc1] | N-(2-(benzo[d]thiazol-2-yl)-3-chlorophenyl)benzamide |
| 32 | 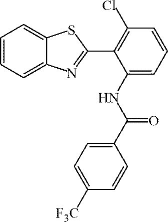 [SMILES: O=C(Nc1cccc(Cl)c1-c1nc2ccccc2s1)c1ccc(C(F)(F)F)cc1] | N-(2-(benzo[d]thiazol-2-yl)-3-chlorophenyl)-4-(trifluoromethyl)benzamide |
| 33 | 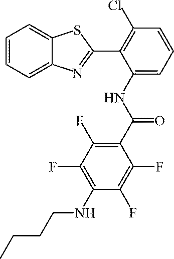 [SMILES: CCCCNc1c(F)c(F)c(C(=O)Nc2cccc(Cl)c2-c2nc3ccccc3s2)c(F)c1F] | N-(2-(benzo[d]thiazol-2-yl)-3-chlorophenyl)-4-(butylamino)-2,3,5,6-tetrafluorobenzamide |
| 34 | 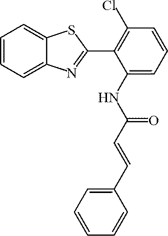 [SMILES: O=C(/C=C/c1ccccc1)Nc1cccc(Cl)c1-c1nc2ccccc2s1] | N-(2-(benzo[d]thiazol-2-yl)-3-chlorophenyl)cinnamamide |
| 35 | 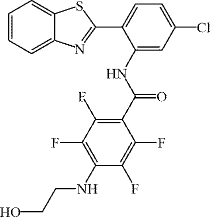 [SMILES: O=C(Nc1cc(Cl)ccc1-c1nc2ccccc2s1)c1c(F)c(F)c(NCCO)c(F)c1F] | N-(2-(benzo[d]thiazol-2-yl)-5-chlorophenyl)-2,3,5,6-tetrafluoro-4-((2-hydroxyethyl)amino)benzamide |

Particularly preferred compounds having formula I incorporated in the polymer matrix of the invention are the compounds 3, 4, 5, 8, 9, 10, 11, 12, 13, 14, 16, 27, 29 and 35 listed in Table 1 above.

Even more particularly preferred compounds having formula I incorporated in the polymer matrix of the invention are the compounds 3, 13, 14 and 27 listed in Table 1 above.

Even more particularly preferred compounds having formula I incorporated in the polymer matrix of the invention are the compounds 13, 14 and 27 listed in Table 1 above.

Within the fluorescent composition according to the invention, the polymer matrix incorporates a quantity of compound having formula I required for the detection of the absorbance and fluorescence properties. The compounds having formula I according to the invention have the advantage of enabling a detection of said properties, even when they are incorporated in small quantities within the polymer matrix. Thus, the quantities of compounds having formula I ranging from 0.005% to 20% by weight with respect to the total weight of the polymer matrix are sufficient for detection, preferably quantities ranging from 0.01% to 15% by weight with respect to the total weight of the polymer matrix and even more preferably, quantities ranging from 0.05% to 10% by weight with respect to the total weight of the polymer matrix. In particular, the quantity of compound having formula I incorporated in the polymer matrix of the fluorescent composition is between 0.005% and 10% by weight with respect to the total weight of the polymer matrix, more specifically between 0.01% and 10%, between 0.01% and 5%, between 0.01% and 1%, between 0.05% and 5%, between 0.05% and 1%, by weight with respect to the total weight of the polymer matrix.

According to a specific embodiment, the fluorescent composition only comprises a polymer matrix integrating a compound having formula I as defined above.

According to a further specific embodiment, the fluorescent composition is essentially formed of a polymer matrix integrating a compound having formula I as defined above. The expression “essentially formed” means according to the present invention that the fluorescent composition is formed by more than 96%, 97%, 98%, or more than 99% of a polymer matrix integrating a compound having formula I.

According to a first alternative embodiment of the invention, the fluorescent composition furthermore comprises a second compound having formula I as defined above, or one of the sub-formulas Ia, Ib and Ic thereof.

According to this alternative embodiment, the quantities of second compound having formula I are from 0.005% to 20% by weight with respect to the total weight of the polymer matrix, preferably quantities ranging from 0.01% to 15% by weight with respect to the total weight of the polymer matrix and even more preferably, quantities ranging from 0.05% to 10% by weight with respect to the total weight of the polymer matrix. In particular, the quantity of the second compound having formula I incorporated in the polymer matrix of the fluorescent composition is between 0.005% and 10% by weight with respect to the total weight of the polymer matrix, more specifically between 0.01% and 10%, between 0.01% and 5%, between 0.01% and 1%, between 0.05% and 5%, between 0.05% and 1%, by weight with respect to the total weight of the polymer matrix.

According to a specific embodiment, the fluorescent composition only comprises a polymer matrix integrating a compound having formula I as defined above and a second compound having formula I as defined above.

According to a specific embodiment, the fluorescent composition also comprises a diffracting grating or a resonant grating.

The fluorescent composition according to the invention comprising a polymer matrix wherein two different compounds having formula I, or one of the sub-formulas Ia, Ib and Ic thereof, as defined above makes it possible to obtain fluorescent compositions with particularly advantageous properties.

Indeed, when two different compounds having formula I, or one of the sub-formulas Ia, Ib and Ic thereof are mixed together, and said compounds emit at different wavelengths, it is then possible to modify the fluorescent colour emitted under UV irradiation. The composition is then coloured only in UV (level 2) and this colour is a mixture of the emissions of the two compounds having formula I.

According to a second alternative embodiment of the invention, the fluorescent composition also comprises a compound of the 4-bora-3a,4a-diaza-s-indacene family, also referred to as “BODIPY”, said BODIPY being incorporated in the polymer matrix. Very specifically, according to this embodiment, the BODIPYs can be compounds having formula III hereinafter:

### [Chem. 6]

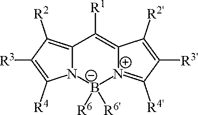

wherein,
R¹ is C1 to C6 alkyl, C5 to C6 cycloalkyl, C5 to C6 heteroalkyl, phenyl, said phenyl group being optionally substituted by one or more groups chosen from C1 to C2 alkyl, hydroxy, R⁵COO— and halogen; preferably R¹ is phenyl optionally substituted by one or more groups chosen from C1 to C2 alkyl; more preferably R¹ is phenyl substituted by one or more groups chosen from C1 to C2 alkyl; more preferably R¹ is phenyl substituted by several groups chosen from C1 to C2 alkyl; more preferably R¹ is phenyl substituted by several methyl groups; more preferably R¹ is 2,4,6-trimethylphenyl;
R² and R^{2′} are independently chosen from hydrogen and C1 to C2 alkyl; preferably R² and R²′ are hydrogen;
R³ and R^{3′} are independently chosen from hydrogen, aryl, heteroaryl, cycloalkyl, alkyl, alkenyl, alkynyl, said aryl, heteroaryl, cycloalkyl, alkyl, alkenyl and alkynyl being optionally substituted by one or more groups chosen from C1 to C4 alkyl, aryl, hydroxy and ferrocene, said aryl groups being optionally substituted by one or more groups chosen from aryl, C1 to C2 alkyl, halogen, hydroxy, dimethylamino, nitro, said aryl being optionally substituted by a C1 to C2 alkyl group; preferably R³ and R^{3′} are hydrogen;
R⁴ and R^{4′} are independently chosen from aryl, heteroaryl, cycloalkyl, alkyl, alkenyl, said aryl, heteroaryl, cycloalkyl, alkyl and alkenyl being optionally substituted by one or more groups chosen from C1 to C3 alkyl, aryl, hydroxy and ferrocene, said aryl group being optionally substituted by one or more groups chosen from aryl, C1 to C2 alkyl, halogen, hydroxy, dimethylamino, nitro, said aryl being optionally substituted by a C1 to C2 alkyl group; preferably R⁴ and R^{4′} are aryl optionally substituted by one or more groups chosen from C1 to C3 alkyl; more preferably R⁴ and R^{4′} are phenyl optionally substituted by one or more groups chosen from C1 to C2 alkyl; more preferably R⁴ and R^{4′} are phenyl substituted by one or more groups chosen from C1 to C2 alkyl; more preferably R⁴ and R^{4′} are phenyl substituted by several groups chosen from C1 to C2 alkyl; more preferably R⁴ and R^{4′} are phenyl substituted by several methyl groups; more preferably R⁴ and R^{4′} are 2,4,6-trimethylphenyl;
R⁵ is C1 to C4 alkyl or C2 to C4 alkenyl.
R⁶ and R^{6′} are independently chosen from halogens, C1 to C4 alkoxy, C2 to C4 alkenyloxy, C1 to C4 alkyl, C2 to C4 alkenyl, CN or aryl, said aryl being optionally substituted by one or more groups chosen from C1 to C2 alkyl, hydroxy, R⁵COO— and halogen; preferably R⁶ and R^{6′} are independently chosen from halogens; more preferably R⁶ and R^{6′} are fluorine atoms.

According to this alternative embodiment, the quantities of compound having formula III are from 0.005% to 20% by weight with respect to the total weight of the polymer matrix, preferably quantities ranging from 0.01% to 15% by weight with respect to the total weight of the polymer matrix and even more preferably, quantities ranging from 0.05% to 10% by weight with respect to the total weight of the polymer matrix. In particular, the quantity of compound having formula III incorporated in the polymer matrix of the fluorescent composition is between 0.005% and 10% by weight with respect to the total weight of the polymer matrix, more specifically between 0.01% and 10%, between 0.01% and 5%, between 0.01% and 1%, between 0.05% and 5%, between 0.05% and 1%, by weight with respect to the total weight of the polymer matrix.

According to a specific embodiment, the fluorescent composition only comprises a polymer matrix integrating a compound having formula I as defined above and a second compound having formula III as defined above.

According to a specific embodiment, the fluorescent composition also comprises a diffracting grating or a resonant grating.

The fluorescent composition according to this second alternative embodiment comprising a polymer matrix wherein a compound having formula I as defined above and a compound having formula III as defined above makes it possible to obtain fluorescent compositions with particularly advantageous properties.

Indeed, when a compound of each family are mixed together and said compounds emit at different wavelengths, it is then possible to modify the fluorescent colour emitted under UV. However, and advantageously, the properties of detecting the fluorescent composition according to level 1 and level 2 with a mobile phone flashlight remain unchanged and are not altered.

The fluorescent composition thus obtained has a colour on a white background and a colour visible for example on a black background and under a mobile phone flashlight obtained from the compound having formula III. Under UV irradiation (level 2), the composition has a third colour obtained from the mixture of the emissions of the compounds having formula I and formula III.

According to a third alternative embodiment of the invention, the fluorescent composition further comprises a fluorescent compound in which the response under UV radiation is checked, which absorbs ultraviolet electromagnetic radiations, particularly between 300 and 400 nm in wavelength, and then re-emits this energy by fluorescence in the visible range, and particularly between 400 and 500 nm.

According to this alternative embodiment, the quantities of fluorescent compound in which the response under UV radiation is checked are from 0.005% to 20% by weight with respect to the total weight of the polymer matrix, preferably quantities ranging from 0.01% to 15% by weight with respect to the total weight of the polymer matrix and even more preferably, quantities ranging from 0.05% to 10% by weight with respect to the total weight of the polymer matrix. In particular, the quantity of fluorescent compound in which the response under UV radiation is checked in the polymer matrix of the fluorescent composition is between 0.005% and 10% by weight with respect to the total weight of the polymer matrix, more specifically between 0.01% and 10%, between 0.01% and 5%, between 0.01% and 1%, between 0.05% and 5%, between 0.05% and 1%, by weight with respect to the total weight of the polymer matrix.

According to a specific embodiment, the fluorescent composition only comprises a polymer matrix integrating a compound having formula I as defined above and a fluorescent compound in which the response under UV radiation is checked.

According to a specific embodiment, the fluorescent composition also comprises a diffracting grating or a resonant grating.

The fluorescent composition according to this second alternative embodiment comprising a polymer matrix wherein a compound having formula I as defined above and a fluorescent compound in which the response under UV radiation is checked makes it possible to obtain fluorescent compositions with particularly advantageous properties.

Thus, the invention secondly relates to the use of a fluorescent composition according to the invention for the securement of a product.

A product according to the present invention can be any type of product capable of receiving said fluorescent composition. The product can thus be a solid or a liquid. It can for example consist of plastic objects such as parts of packaging materials, luxury products such as leatherware, cosmetic products, paintings or documents. Preferably, the products are documents.

The term document refers particularly to an assembly formed by a substrate and information. The substrate can be of different types, take different forms, and can optionally comprise a polymer or a polymer mixture. This substrate can for example be formed entirely or partially of a polymer material. By way of example of a document, mention can particularly be made of identity documents such as passports, identity cards, driving licences or health cards, but also fiduciary documents such as banknotes and cheques, or administrative documents such as registration certificates for example. The document can therefore be presented in paper, booklet or card form and the information can equally well be printed and/or etched.

The expression “securement of a product” means according to the present invention that the fluorescent composition is integrated on the product or in the product to be secured at any time of the design thereof. Thus, the fluorescent composition can equally well be used for the securement of the product during the manufacture thereof, or be applied or integrated retrospectively thereto. For example, within the scope of the securement of a document type product, the fluorescent composition can be applied retrospectively on all or part of the document. This point is developed further in the description.

In any case, the products are secured by the use of a fluorescent composition comprising a polymer matrix incorporating a compound having formula I described above, a mixture of two compounds having formula I, or a mixture of a compound having formula I and a compound having formula III, and can be authenticated thanks to the properties and the effects provided by the fluorescent composition.

Indeed, the products secured according to the invention can be authenticated thanks to the unique combination of the absorbed wavelength and the specific fluorescence of the fluorescent composition. Thus, only an authentic product will simultaneously have the correct absorption and fluorescence emission properties. Authentication according to the present invention means the verification of the authenticity of a product through the detection of the fluorescent composition or securement means incorporated therein. In the case of the use of a composition comprising a compound having formula I and a compound having formula III, this detection of the presence or absence or colouring or fluorescence thus makes it possible to authenticate the product in question or not. A product is therefore authentic when a detection reveals the presence of the fluorescent composition, as opposed to a non-authentic product for which the detection will not reveal the fluorescent composition. The products secured according to the invention by means of a fluorescent composition can be authenticated on the three security levels described hereinafter thanks to the sole presence of said fluorescent composition comprising a compound having formula I and a compound having formula III.

Indeed, in the case of the use of a composition comprising a compound having formula I and a compound having formula III, the compounds having formula III contained in the fluorescent composition can have an absorption band in the visible range and the colour perceived by the naked eye will correspond to the complementary colour of the absorbed colour. For example a compound absorbing around 500-520 nm, which corresponds to a green/blue colour, will appear in orange/red tones to the naked eye. This property thus makes it possible to obtain a level 1 securement.

In relation to the fluorescence properties, the compounds having formula I according to the invention all have excitation bands in the ultraviolet (UV) range. They can therefore be excited by means in particular of a UV or LED lamp emitting between 100 nm and 400 nm which makes it possible to obtain a level 2 securement. This property makes it possible to obtain an activation/deactivation (on/off) effect which therefore corresponds to the display of a change of colour following a stimulation of the fluorescence of the fluorescent composition, particularly by an LED or UV type light source.

Finally, the emission wavelength could be determined using a single-grating low-resolution spectrofluorometer or fluorometer (detection by photodiode or photomultiplier tube), which gives the security elements according to the present invention a security level 3.

Thus, the products and particularly the secured documents according to the present invention will be, thanks to the combination of the absorption and fluorescence properties, detectable on levels 2 and 3 in the case of the use of a composition comprising a compound having formula I or a mixture of two compounds having formula I, and on the 3 securement levels in the case of the use of a composition comprising a compound having formula I and a compound having formula III.

The fluorescent composition according to the present invention can be presented in several forms which are adapted by a person skilled in the art according to the product to be secured. For example, if the product is a document, the fluorescent composition can be in the form of a layer, a set of layers or a film.

According to a specific embodiment, the fluorescent composition is used in the form of a layer or a set of layers prepared using techniques known to a person skilled in the art such as for example rolling, extrusion, calendering, or calendering extrusion. These techniques will be chosen according to the polymer matrix used. By way of example, if the matrix is made of polycarbonate or thermoplastic polyurethane, shaping by calendering extrusion is preferred. Also by way of example, if the matrix is made of polypropylene, the principle of pumping extrusion is preferred, in particular the principle of pumping extrusion with bi-drawing. A set of layers according to the present invention can for example be obtained by rolling two or several layers of polymer matrix each incorporating one or more fluorescent compositions. Such a layer or set of layers finds a particularly advantageous application in the securement of documents, and more specifically identity, fiduciary or administrative documents.

According to this specific embodiment, the layer or set of layers is in card form. Examples of cards are particularly business cards, bank cards, or any other type of card made of polymer matrix. In this case, the fluorescent composition according to the invention forms the substrate of the document per se. The cards may for example be obtained by rolling several layers of polymer of which at least one is the fluorescent composition according to the present invention.

Particularly advantageously, in the case of the use of a composition comprising a compound having formula I, the layer or the set of layers is transparent which makes it possible to obtain, in addition to the effects described above, the following effects:
waveguide effect: the presence of grooves in the layer or the set of layers, generates a different diffraction index which stimulates the fluorescence of the secured layer. Thus, the colour observed at the groove is different from that observed on the rest of the layer or the set of layers. Under UV irradiation, this represents level 2 security.
side-effect: this effect corresponds to the observation of a complementary colour of the absorbed colour which is different on the sides of the layer or the set of layers, from that observed on the faces. Under UV irradiation, this represents level 2 security.

Particularly advantageously, in the case of the use of a composition comprising a mixture of a compound having formula I and a compound having formula III, the layer or the set of layers is transparent which makes it possible to obtain, in addition to the effects described above, the following effects:
waveguide effect: the presence of grooves in the layer or the set of layers, generates a different diffraction index which stimulates the fluorescence of the secured layer. Thus, the colour observed at the groove is different from that observed on the rest of the layer or the set of layers. This represents level 1 security.
switch-colour effect: this effect corresponds to a change of colour when superimposing the layer or the set of layers on a contrasting background (such as dark, particularly black, background, or light, particularly white, background. The complementary colour of the absorbed colour on a light background and the fluorescence colour on a dark background is thus observed. This represents level 1 security.
side-effect: this effect corresponds to the observation of a complementary colour of the absorbed colour which is different on the sides of the layer or the set of layers, from that observed on the faces. This represents level 1 security.
shadow effect: this effect, linked with the presence of the compound having formula III, corresponds, when fluorescence is stimulated by an LED or UV type light and when observing on a light, particularly white, background, the display of both the fluorescence colour at the layer and the projection of the complementation colour of the absorbed colour on the light background. This represents level 2 security.

Advantageously, a layer particularly has a thickness ranging from 0.050 mm to 0.800 mm, preferably a thickness ranging from 0.200 mm to 0.600 mm, such as for example a thickness of approximately 0.400 mm. When the layer has a thickness less than 0.100 mm, it is also referred to as a film.

According to a specific embodiment, the fluorescent composition used for the securement of a product only comprises a polymer matrix integrating a compound having formula I as defined above.

According to a further specific embodiment, the fluorescent composition used for the securement of a product is essentially formed of a polymer matrix integrating a compound having formula I as defined above. The expression “essentially formed” means according to the present invention that the fluorescent composition is formed by more than 96%, 97%, 98%, or more than 99% of a polymer matrix integrating a compound having formula I.

According to a further specific embodiment, the fluorescent composition used for the securement of a product is formed by approximately 50% of a polymer matrix integrating a compound having formula I as defined above.

According to a specific embodiment, the fluorescent composition used for the securement of a product only comprises a polymer matrix integrating a mixture of two different compounds having formula I as defined above.

The fluorescent composition thus obtained has a colour obtained from the mixture of the colours of each of the two compounds having formula I and which is only visible in UV irradiation (level 2).

According to a further specific embodiment, the fluorescent composition used for the securement of a product only comprises a polymer matrix integrating a compound having formula I and a compound having formula III as defined above.

Indeed, when a compound having formula I and a compound having formula III are mixed together and said compounds emit at different wavelengths, it is then possible to modify the fluorescent colour emitted under UV irradiation. However, and advantageously, the properties of detecting the fluorescent composition according to level 1 and level 2 with a mobile phone flashlight remain unchanged and are not altered.

The fluorescent composition thus obtained has three colours, a first which is visible on a white background (level 1), a second which is visible for example on a black background or under a mobile phone flashlight (level 1), and a third under UV irradiation (level 2).

Thus, the use of a composition comprising a mixture of compound having formula I and a compound having formula III makes it possible to complexify level 2.

According to a specific embodiment, the fluorescent composition is used in the form of a fluorescent ink. According to this embodiment, the fluorescent ink is an ink adapted for printing, in particular for screen printing, offset printing, flexography, heliography, ink-jet printing, digital printing, copperplate printing, and 3D printing, preferably for offset printing, and ink-jet printing. Completely surprisingly, the inventors advantageously observed that the fluorescent inks according to the invention could be used in printing without causing clogging of the print heads.

According to a specific embodiment, the fluorescent composition is used in the form of a water-based ink.

According to a further specific embodiment, the fluorescent composition is used in the form of a fluorescent varnish.

According to a specific embodiment, the fluorescent composition is used in the form of a film, i.e. a layer having a thickness less than 0.100 mm, particularly ranging from 0.050 mm to 0.100 mm, which is used to roll both faces of a document, particularly of an identity, fiduciary or administrative document. In an alternative embodiment of this embodiment, such a film is only applied to one of the two faces of a document, particularly an identity document. In another alternative embodiment of the embodiment, such a film is only applied to a portion of one of the two faces of a document, particularly an identity document. In another alternative embodiment of this embodiment, such a film is only applied to a portion of each of the two faces of a document, particularly an identity document.

According to a further specific embodiment, the fluorescent composition is shaped in the form of a fibre. This shaping can be performed using techniques conventionally implemented for obtaining fibres, said fibres being either woven fibres or nonwoven fibres.

According to this embodiment, the fibres are preferably obtained with the molten process spinning technique, via an extrusion-spinning method. The manufacture of fibres by means of the molten process spinning method firstly consists of melting the mixture of polymer and fluorescent compound in an extruder. The molten material is then sent under pressure through a die consisting of a multitude of heads. At the die output, the filaments are air-cooled, stretched then coiled on a substrate. Generally, a sizing product can be applied to the lower portion of the spinning shaft.

According to an embodiment, the compound having formula I, the mixture of two compounds having formula I or the mixture of a compound having formula I and a compound having formula III can be integrated in a polymer matrix without using extrusion, particularly by yarn impregnation.

The shape of the fluorescent fibre obtained according to the extrusion-spinning methods can particularly be determined by the shape of the die heads. Thus, the fibre can particularly have a cylindrical, trilobate, octolobate, hollow or multiple hollow shape. The modification of the shape of the fibres can be advantageous in that it makes it possible to modify the visual effects on a macroscopic scale. Indeed, the discontinuity of a section or of the refractive index of the light inside the fibre can modify the light transmission and therefore, the effects observed on a macroscopic scale.

According to a further specific embodiment, several fluorescent compositions are used to secure the same product, the fluorescent compositions being different from one another at least by the nature of the compound having formula I, the mixture of two compounds having formula I or the mixture of a compound having formula I and a compound having formula III, integrated in the polymer matrix. This embodiment makes it possible advantageously to better secure the product in question.

According to a further specific embodiment, the polymer matrix is a photopolymerisable resin and the fluorescent composition also comprises a polar solvent to facilitate the integration between the resin and the compound having formula I, the mixture of two compounds having formula I or the mixture of a compound having formula I and a compound having formula III. The fluorescent composition obtained thus finds a very specific application in certain 3D printing techniques, for example for producing holograms, said holograms then having a higher security level.

The invention thirdly relates to a method for the securement of a product comprising the following steps of:
preparing a fluorescent composition as defined above,
securement by applying said fluorescent composition prepared in the preceding step, on at least one of the portions of said product.

The first step therefore consists of obtaining a fluorescent composition as defined above.

According to a specific embodiment, the first step of the securement method consists of preparing a fluorescent composition only comprising a polymer matrix integrating a compound having formula I as defined above.

According to a further specific embodiment, the first step of the securement method consists of preparing a fluorescent composition essentially formed of a polymer matrix integrating a compound having formula I as defined above. The expression “essentially formed” means according to the present invention that the fluorescent composition is formed by more than 96%, 97%, 98%, or more than 99% of a polymer matrix integrating a compound having formula I.

According to a specific embodiment, the first step of the securement method consists of preparing a fluorescent composition only comprising a polymer matrix integrating two different compounds having formula I as defined above.

According to a specific embodiment, the first step of the securement method consists of preparing a fluorescent composition only comprising a polymer matrix integrating a compound having formula I as defined above and a compound having formula III as defined above.

According to a specific embodiment, the fluorescent composition is a fluorescent ink. According to this embodiment, the fluorescent ink is obtained from an ink known to a person skilled in the art, said inks comprising a polymer matrix, in which a compound having formula I, a mixture of two different compounds having formula I, or a mixture of a compound having formula I and a compound having formula III is incorporated, so as to obtain the fluorescent ink. According to this embodiment, the securement step is advantageously carried out by printing the fluorescent ink on the product to be secured, such as for example a document.

According to a specific embodiment, the fluorescent composition is a fluorescent varnish. According to this embodiment, the securement step can for example be carried out by coating or varnishing said fluorescent varnish on the product to be secured.

The securement step on all or part of the product to be secured is adapted by a person skilled in the art according to the product to be secured but also the form of said composition.

The securement step consists of integrating the fluorescent composition in the product to be secured. Thus, the securement step can equally well be carried out during the manufacture of the product, and carried out retrospectively thereto. For example, within the scope of the securement of a document type product, the securement step can be carried out on the finished product. Furthermore, this step can be reproduced several times on the same product in order to increase the securement level of the product.

The securement step can be carried out according to techniques known to a person skilled in the art such as for example by rolling, printing, weaving, varnishing, lacquering, bonding, coating or impregnation.

According to a specific embodiment, the fluorescent composition according to the invention comprising a compound having formula I and a compound having formula III is applied by coating on a reflective surface or a metallised surface. The product is then secured by applying the assembly consisting of the reflective or metallised layer coated with the fluorescent composition. The metallised surfaces are layers known to a person skilled in the art, it can consist for example of an aluminium metal layer.

Advantageously, according to this embodiment, the fluorescence properties of the fluorescent composition interact with the reflective appearance of the reflective layer and this makes it possible to obtain specific visual effects for the securement of a product. At equivalent concentration, a reflective surface exalts the light intensity with respect to a nonreflective surface.

The securement method according to the invention can also comprise a step of shaping the fluorescent composition before the securement step. The shaping step can be carried out according to the techniques known to a person skilled in the art which make it possible for example to obtain a layer, a set of layers, a film or fibres. This shaping step can thus facilitate the subsequent securement step by application.

According to a specific embodiment, the first step of the method consists of preparing several fluorescent compositions which differ by the nature of the compound having formula I integrated in the polymer matrix. According to this embodiment, the securement step consists of applying in a simultaneous or deferred manner the prepared compositions and thus increase the security level given to the product.

The method according to the invention can be used to secure any type of product capable of receiving said fluorescent composition. It can for example consist of plastic objects such as parts of packaging materials, luxury products such as leatherware, or documents. Preferably, the products secured according to the method described are documents.

According to a specific embodiment, the product to be secured is a document such as an identity, fiduciary or administrative document. According to this embodiment, the application of the fluorescent securement composition can be carried out on at least a portion of a face of the product. According to an alternative embodiment of this embodiment, the fluorescent securement composition is shaped from a film before being hot- or cold-rolled on all of the faces of the document. In a further alternative embodiment, the film is only applied on one of the faces of a document. In another alternative embodiment, the film is only applied to a portion of one of the two faces of a document and in another alternative embodiment, the film is only applied to a portion of each of the faces of a document.

According to a further specific embodiment, the polymer matrix of the fluorescent composition also incorporates a compound having formula II defined above.

The invention further relates to a compound having formula II:

### [Chem. 2]

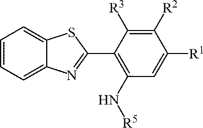

wherein,
R¹, and R² and R³ are independently chosen from hydrogen, C1-C6-alkyl, C3-C6-cycloalkyl, aryl, vinyl, ethynyl, halogen, —NO₂, —NH₂, —NHR⁴, —N(R⁴)₂, —N⁺(R⁴)₃, —NHCOR⁴, —CHO, —C(O)OH, —C(O)OR⁴, —CF₃, C1-C6-alkoxy, aryloxy, —SH, —SO₃H, —SR⁴; preferably R¹, and R² and R³ are independently chosen from hydrogen, C1-C6-alkyl, C1-C6-alkoxy, halogen and —NO₂; preferably R¹, and R² and R³ are independently chosen from hydrogen, C1-C4-alkyl, C1-C4-alkoxy, halogen and —NO₂; more preferably R¹, and R² and R³ are independently chosen from hydrogen, C1-C2-alkyl, C1-C2-alkoxy, chloro, fluoro and —NO₂; more preferably R¹, and R² and R³ are independently chosen from hydrogen, methyl, methoxy, chloro, fluoro and —NO₂; even more preferably R¹, and R² and R³ are independently chosen from hydrogen, methyl, methoxy, chloro and fluoro;
R⁴ is chosen from C1-C6-alkyl, C1-C6-cycloalkyl and aryl;
R⁵ is chosen from C1-C6-alkyl, hydroxy-C1-C6-alkyl, C1-C6-alkyloxycarbonyl, di-C1-C6-alkylaminothionyl, C1-C6-alkylsulfonyl, cinnamoyl, benzoyl, 2,3,4,5,6-pentahalogenobenzoyl and 2,3,5,6-tetrahalogenobenzoyl, optionally substituted by C1-C6-alkyl, C1-C6-alkoxy, trifluoro-C1-C6-alkyl, C1-C6-alkylamino or hydroxy-C1-C6-alkylamino; preferably R⁵ is chosen from cinnamoyl, benzoyl, 2,3,4,5,6-pentahalogenobenzoyl and 2,3,5,6-tetrahalogenobenzoyl, optionally substituted by C1-C6-alkyl, C1-C6-alkoxy, trifluoro-C1-C6-alkyl, C1-C6-alkylamino or hydroxy-C1-C6-alkylamino; more preferably R⁵ is chosen from cinnamoyl, benzoyl, 2,3,4,5,6-pentahalogenobenzoyl and 2,3,5,6-tetrahalogenobenzoyl, optionally substituted by C1-C4-alkyl, C1-C4-alkoxy, trifluoro-C1-C4-alkyl, C1-C4-alkylamino or hydroxy-C1-C4-alkylamino; more preferably R⁵ is chosen from cinnamoyl, benzoyl, 2,3,4,5,6-pentahalogenobenzoyl and 2,3,5,6-tetrahalogenobenzoyl, optionally substituted by C1-C2-alkyl, C1-C2-alkoxy, trifluoro-C1-C2-alkyl, C1-C4-alkylamino or hydroxy-C1-C2-alkylamino; even more preferably R⁵ is chosen from cinnamoyl, benzoyl, 2,3,4,5,6-pentahalogenobenzoyl and 2,3,5,6-tetrahalogenobenzoyl, optionally substituted by methyl, methoxy, trifluoromethyl, butylamino or hydroxyethylamino; even more preferably R⁵ is chosen from cinnamoyl, benzoyl, 2,3,4,5,6-pentahalogenobenzoyl and 2,3,5,6-tetrahalogenobenzoyl, optionally substituted by trifluoromethyl, butylamino or hydroxyethylamino; for example R⁵ is chosen from benzoyl, 4-(trifluoromethyl)benzoyl, 3,5-bis(trifluoromethyl)benzoyl, 2,3,4,5,6-pentafluorobenzoyl, 4-(butylamino)-2,3,5,6-tetrafluorobenzoyl, cinnamoyl and 4-((2-hydroxyethyl)amino)benzoyl; and with the condition that:
R¹, R² and R³ are not all three hydrogen when R⁵ is cinnamoyl, or benzoyl optionally substituted by methyl, methoxy, chloro or trifluoromethyl;
R¹ is not methyl when R⁵ is benzoyl;
R² is not methyl when R⁵ is benzoyl; and
R³ is not methyl, methoxy or chloro when R⁵ is benzoyl.

In an embodiment, the compounds having formula II are those having formula IIa:

### [Chem. 7]

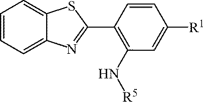

wherein R¹ and R⁵ are as defined in formula II.

Preferred compounds having formula IIa are those wherein R¹ is methoxy or chloro.

In an embodiment, the compounds having formula II are those having formula IIb:

### [Chem. 8]

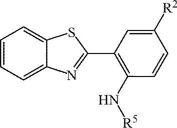

wherein R² and R⁵ are as defined in formula II.

Preferred compounds having formula IIa are those wherein R² is chosen from methyl, methoxy, fluoro and NO₂.

In an embodiment, the compounds having formula II are those having formula IIc:

### [Chem. 9]

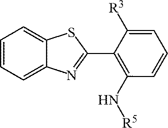

wherein R³ and R⁵ are as defined in formula II.

Preferred compounds having formula III are those wherein R³ is chloro.

Particularly preferred compounds having formula II of the invention are those listed in Table 2 hereinafter:

TABLE 2

| Compound | Structure | Name |
|---|---|---|
| 3 | 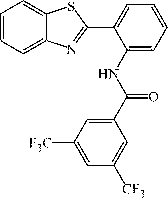 [SMILES: O=C(Nc1ccccc1-c1nc2ccccc2s1)c1cc(C(F)(F)F)cc(C(F)(F)F)c1] | N-(2-(benzo[d]thiazol-2-yl)phenyl)-3,5-bis(trifluoromethyl)benzamide |
| 4 | 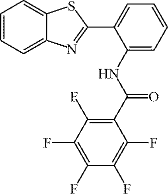 [SMILES: O=C(Nc1ccccc1-c1nc2ccccc2s1)c1c(F)c(F)c(F)c(F)c1F] | N-(2-(benzo[d]thiazol-2-yl)phenyl)-2,3,4,5,6-pentafluorobenzamide |
| 5 | 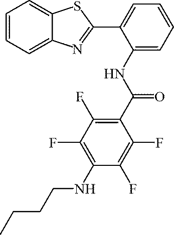 [SMILES: CCCCNc1c(F)c(F)c(C(=O)Nc2ccccc2-c2nc3ccccc3s2)c(F)c1F] | N-(2-(benzo[d]thiazol-2-yl)phenyl)-4-(butylamino)-2,3,5,6-tetrafluorobenzamide |
| 8 | 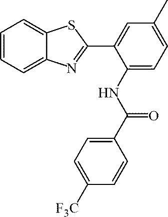 [SMILES: Cc1ccc(NC(=O)c2ccc(C(F)(F)F)cc2)c(-c2nc3ccccc3s2)c1] | N-(2-(benzo[d]thiazol-2-yl)-4-methylphenyl)-4-(trifluoromethyl)benzamide |
| 9 | 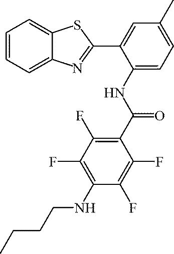 [SMILES: CCCCNc1c(F)c(F)c(C(=O)Nc2ccc(C)cc2-c2nc3ccccc3s2)c(F)c1F] | N-(2-(benzo[d]thiazol-2-yl)-4-methylphenyl)-4-(butylamino)-2,3,5,6-tetrafluorobenzamide |
| 10 | 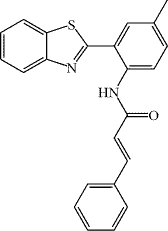 [SMILES: Cc1ccc(NC(=O)/C=C/c2ccccc2)c(-c2nc3ccccc3s2)c1] | N-(2-(benzo[d]thiazol-2-yl)-4-methylphenyl)cinnamamide |
| 11 | 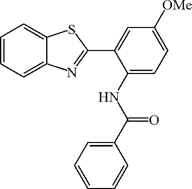 [SMILES: COc1ccc(NC(=O)c2ccccc2)c(-c2nc3ccccc3s2)c1] | N-(2-(benzo[d]thiazol-2-yl)-4-methoxyphenyl)benzamide |
| 12 | 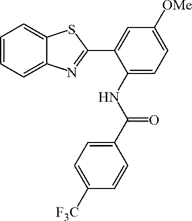 [SMILES: COc1ccc(NC(=O)c2ccc(C(F)(F)F)cc2)c(-c2nc3ccccc3s2)c1] | N-(2-(benzo[d]thiazol-2-yl)-4-methoxyphenyl)-4-(trifluoromethyl)benzamide |
| 13 | 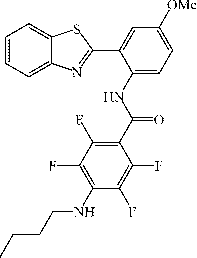 [SMILES: CCCCNc1c(F)c(F)c(C(=O)Nc2ccc(OC)cc2-c2nc3ccccc3s2)c(F)c1F] | N-(2-(benzo[d]thiazol-2-yl)-4-methoxyphenyl)-4-(butylamino)-2,3,5,6-tetrafluorobenzamide |
| 14 | 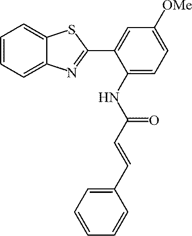 [SMILES: COc1ccc(NC(=O)/C=C/c2ccccc2)c(-c2nc3ccccc3s2)c1] | N-(2-(benzo[d]thiazol-2-yl)-4-methoxyphenyl)cinnamamide |
| 16 | 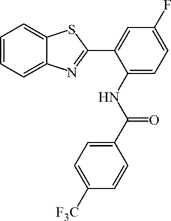 [SMILES: O=C(Nc1ccc(F)cc1-c1nc2ccccc2s1)c1ccc(C(F)(F)F)cc1] | N-(2-(benzo[d]thiazol-2-yl)-4-fluorophenyl)-4-(trifluoromethyl)benzamide |
| 27 | 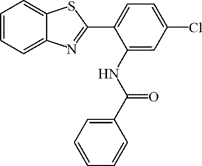 [SMILES: O=C(Nc1cc(Cl)ccc1-c1nc2ccccc2s1)c1ccccc1] | N-(2-(benzo[d]thiazol-2-yl)-5-chlorophenyl)benzamide |
| 29 | 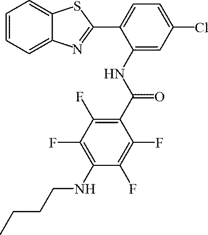 [SMILES: CCCCNc1c(F)c(F)c(C(=O)Nc2cc(Cl)ccc2-c2nc3ccccc3s2)c(F)c1F] | N-(2-(benzo[d]thiazol-2-yl)-5-chlorophenyl)-4-(butylamino)-2,3,5,6-tetrafluorobenzamide |
| 35 | 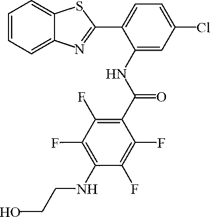 [SMILES: O=C(Nc1cc(Cl)ccc1-c1nc2ccccc2s1)c1c(F)c(F)c(NCCO)c(F)c1F] | N-(2-(benzo[d]thiazol-2-yl)-5-chlorophenyl)-2,3,5,6-tetrafluoro-4-((2-hydroxyethyl)amino)benzamide |

### Definitions

The definitions and explanations hereinafter relate to the terms and expressions as used in the present application, comprising the description as well as the claims.

For the description of the compounds according to the invention, the terms and expressions used should, unless specified otherwise, be interpreted according to the definitions hereinafter.

The term “alkyl”, alone or as part of another group, denotes a hydrocarbon radical having formula CₙH₂ₙ₊₁ wherein n is an integer greater than or equal to 1. The preferred alkyl groups are linear or branched C1 to C6 alkyl groups.

The term “alkenyl” denotes a linear or branched unsaturated alkyl group, comprising one or more carbon-carbon double bonds. Suitable alkenyl groups comprise from 2 to 6 carbon atoms, preferably from 2 to 4 carbon atoms and more preferably 2 or 3 carbon atoms. Non-limiting examples of alkenyl groups are ethenyl (vinyl), 2-propenyl (allyl), 2-butenyl and 3-butenyl, ethenyl and 2-propenyl being preferred.

The term “cycloalkyl”, alone or as part of another group, denotes a saturated mono-, di- or tri-cyclic hydrocarbon radical having 3 to 12 carbon atoms, particularly 5 to 10 carbon atoms, more specifically 6 to 10 carbon atoms. Suitable cycloalkyl radicals comprise, without being limited thereto, cyclopropyl, cyclobutyl, cyclopentyl, cyclohexyl, cycloheptyl, cyclooctyl, norbornyl, adamantyl, particularly adamant-1-yl and adamant-2-yl, 1-decalinyl. Preferred cycloalkyl groups comprise cyclopropyl, cyclohexyl, and cycloheptyl. A particularly preferred cycloalkyl group is cyclohexyl.

The term “aryl”, alone or as part of another group, denotes a polyunsaturated aromatic hydrocarbon radical having a single ring (phenyl) or several aromatic rings condensed together (for example naphthyl) typically containing 5 to 12 atoms preferably 6 to 10, wherein at least one of the rings is aromatic. Preferred aryl groups comprise phenyl, naphthyl, anthracenyl, phenanthracenyl, pyrenyl. A particularly preferred aryl group is phenyl.

The term “heteroaryl”, alone or as part of another group, denotes, without being limited thereto, aromatic rings or cyclic systems containing one to two rings condensed together, typically containing 5 to 12 atoms, wherein at least one of the rings is aromatic, and wherein one or more carbon atoms in one or more of these rings are replaced by oxygen, nitrogen and/or sulphur atoms, the nitrogen and sulphur heteroatoms optionally being oxidised and the nitrogen heteroatoms optionally being quaternised. Preferred but non-limiting heteroaryl groups are pyridinyl, pyrrolyl, furanyl, thiophenyl. Particularly preferred heteroaryl groups are thiophenyl and pyridinyl.

The term “halo”, alone or as part of another group, denotes fluoro, chloro, bromo, or iodo. The preferred halo are chloro and fluoro, fluoro being particularly preferred.

The term “haloalkyl”, alone or as part of another group, denotes an alkyl radical as defined above wherein one or more hydrogen atoms are replaced by a halo group as defined above. The haloalkyl radicals according to the present invention can be linear or branched, and comprise, without being limited thereto, radicals having formula CₙF₂ₙ₊₁ wherein n is an integer greater than or equal to 1, preferably an integer between 1 and 10. Preferred haloalkyl radicals comprise trifluoromethyl, difluoromethyl, fluoromethyl, pentafluoroethyl, heptafluoro-n-propyl, nonafluoro-n-butyl, 1,1,1-trifluoro-n-butyl, 1,1,1-trifluoro-n-pentyl and 1,1,1-trifluoro-n-hexyl, trifluoromethyl being particularly preferred.

The present invention will be better understood with reference to the following examples. These examples are representative of certain embodiments of the invention and in no way restrict the scope of the invention. The figures serve to illustrate the experimental results.

### EXAMPLES

### Chemical Synthesis

All the temperatures are expressed in °C and all the reactions were performed at ambient temperature (AT), unless specified otherwise.

The reactions were monitored by thin layer chromatography (TLC) conducted on ready-to-use aluminium sheets coated with a silica gel and a UV254 fluorescence indicator (Kieselgel® 60 F254 Merck of 0.2 mm thickness) or equivalent.

The NMR analyses were conducted on a Bruker 300 MHz, 400 MHz or 600 MHz spectrometer. The spectra are recorded in solution in deuterated chloroform (CDCl₃). The chemical shifts are given in ppm, monitored for multiplicity proton spectra, where s, sl, d, t, q, dd, td and m respectively denote the singlets, broad singlets, doublets, triplets, quadruplets, doublets of doublets, triplets of doublets and multiplets (or low-resolution masses). The multiplicities are followed where applicable by the coupling constant value annotated J and expressed in Hertz (Hz).

The HRMS analyses were conducted in positive electrospray ionisation mode (ESI +).

The solvents, reagents, and starting materials were purchased from well-known chemical product suppliers such as Sigma Aldrich, Acros Organics, Fluorochem, Eurisotop, VWR International, Sopachem and Polymer. The solvents, unless specified otherwise, were purified by distillation before use. The reagents and the starting materials, unless specified otherwise, were used without additional purifications.

The following abbreviations were used:
HRMS: high-resolution mass spectrometry,
NMR: nuclear magnetic resonance,
AT: ambient temperature,
THF: tetrahydrofuran.

### Compound 3: N-(2-(benzo[d]thiazol-2-yl)phenyl)-3,5-bis(trifluoromethyl)benzamide

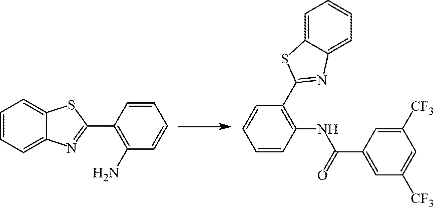 [SMILES: Nc1ccccc1-c1nc2ccccc2s1 ; O=C(Nc1ccccc1-c1nc2ccccc2s1)c1cc(C(F)(F)F)cc(C(F)(F)F)c1]

In a 50 mL round-bottomed flask equipped with a stirrer and a temperature indicator, 2-(1,3-benzothiazol-2-yl)aniline (0.5 g) and pyridine (0.9 mL) were introduced into tetrahydrofuran (7 mL). 3,5-bis (trifluoromethyl)benzoyl chloride (0.44 g) was then added and the solution was heated to 60° C. After stirring for approximately 45 minutes at 60° C., the reaction mixture was cooled to ambient temperature. 10 mL of water was added and the mixture was filtered, the filter residue was washed with 2 × 25 mL of water, 25 mL of ethanol and dried to give compound 3 (0.87 g).

¹H NMR (CDCl₃): δ 13.50 (1H, s), 9.02 (1H, dd, J = 5.6, 0.8 Hz), 8.66 (2H, s), 8.15 (1H, s), 8.07 (1H, d, J = 5.6 Hz), 7.97 (1H, dd, J = 5.2, 1.2 Hz), 7.96 (1H, d, J = 5.6 Hz), 7.6 - 7.55 (2H, m), 7.48 (1H, td, J = 5, 0.8 Hz), 7.29 (1H, td, J = 5.2, 1.2 Hz) HRMS (ESI+) m/z 467.12 (467.41 calculated for C₂₂H₁₂F₆N₂OS+H⁺ [M +H]⁺).

### Compound 4: N-(2-(benzo[d]thiazol-2-yl)phenyl)-2,3,4,5,6-pentafluorobenzamide

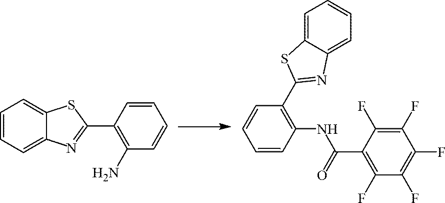 [SMILES: Nc1ccccc1-c1nc2ccccc2s1 ; O=C(Nc1ccccc1-c1nc2ccccc2s1)c1c(F)c(F)c(F)c(F)c1F]

The same procedure as that described above for the preparation of compound 3 was used with 1.0 g of 2-(1,3-benzothiazole-2-yl)aniline, 1.8 mL of pyridine, 13 mL of THF and 0.7 mL of pentafluorobenzoyl chloride to give compound 4 (1.51 g).

¹H NMR (CDCl₃): δ 13.46 (1H, s), 8.94 (1H, dd, J = 5.6, 0.4 Hz), 7.95-7.93 (2H, m), 7.74 (1H, d, J = 5.6 Hz), 7.58 (1H, td, J = 5.2, 0.4 Hz), 7.53 (1H, td, J = 5.2, 0.8 Hz), 7.46 (1H, td, J = 5.2, 0.4 Hz), 7.30 (1H, td, J = 5.2, 0.8 Hz).

HRMS (ESI+) m/z 421.14 (421.36 calculated for C₂₀H₉F₅N₂OS+H⁺ [M +H]⁺).

### Compound 5: N-(2-(benzo[d]thiazol-2-yl)phenyl)-4-(butylamino)-2,3,5,6-tetrafluorobenzamide

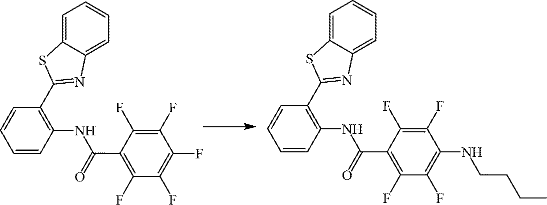 [SMILES: O=C(Nc1ccccc1-c1nc2ccccc2s1)c1c(F)c(F)c(F)c(F)c1F ; CCCCNc1c(F)c(F)c(C(=O)Nc2ccccc2-c2nc3ccccc3s2)c(F)c1F]

In a 100 mL round-bottomed flask equipped with a stirrer and a temperature indicator, potassium carbonate (4.6 g), N-(2-(benzo[d]thiazol-2-yl)phenyl)-2,3,4,5,6-pentafluorobenzamide (7.00 g) and dimethylformamide (25 mL) were introduced. The resulting mixture was stirred at ambient temperature. Butylamine (2.43 g) was then added and the mixture was heated to 95° C. After 1 hour, the reaction mixture was cooled to ambient temperature, filtered and the filter residue was washed with 2 × 30 mL of water. Then, in a 50 mL round-bottomed flask equipped with a stirrer and a temperature indicator, the raw products and petroleum ether (25 mL) were introduced. The resulting mixture was heated to 50° C., after 45 minutes, the reaction mixture was cooled to ambient temperature, and the mixture was filtered and the filter residue was washed with 2 × 15 mL of petroleum ether to give compound 5 (8.7 g).

¹H NMR (CDCl₃): δ 13.22 (1H, s), 9.99 (1H, d, J = 5.6 Hz), 7.93 (1H, d, J =5.2 Hz), 7.91 (1H, dd, J = 5.2, 0.8 Hz), 7.84 (1H, d, J = 5.6 Hz), 7.54 (1H, td, J = 5.2, 0.8 Hz), 7.51 (1H, td, J = 5.2, 0.4 Hz), 7.44 (1H, td, J = 5.2, 0.4 Hz), 7.42 (1H, td, J = 5.2, 0.4 Hz), 4.14 (1H, bs), 3.55 (2H, t, H = 4.8 Hz), 1.68 (2H, q, J = 4.8 Hz), 1.48 (2H, h, J = 5.2 Hz), 1.02 (3H, t, J = 5.2 Hz).

HRMS (ESI+) m/z 474.21 (474.49 calculated for C₂₄H₁₉F₄N₃OS+H⁺ [M +H]⁺).

### Compound 8: N-(2-(benzo[d]thiazol-2-yl)-4-methylphenyl)-4-(trifluoromethyl)benzamide

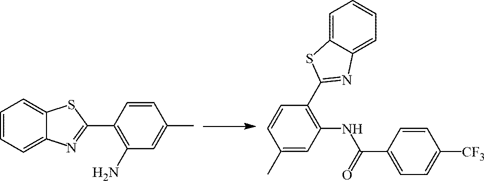 [SMILES: Cc1ccc(-c2nc3ccccc3s2)c(N)c1 ; Cc1ccc(-c2nc3ccccc3s2)c(NC(=O)c2ccc(C(F)(F)F)cc2)c1]

The same procedure as that described above for the preparation of compound 3 was used with 0.61 g of 2-(1,3-benzothiazole-2-yl)-5-methylaniline, 0.8 mL of pyridine, 8 mL of THF and 0.75 mL of trifluoromethylbenzoyl chloride to give compound 8 (0.53 g).

¹H NMR (CDCl₃): δ 13.47 (1H, s), 8.87 (1H, d, J = 3.2 Hz), 8.37 (2H, d, J = 5.6 Hz), 7.96 (2H, t, J = 5.2 Hz), 7.88 (2H, d, J = 5.6 Hz), 7.82 (1H, d, J = 5.2 Hz), 7.59 (1H, t, J = 5.2 Hz), 7.47 (1H, t, J = 5.2 Hz), 7.06 (1H, d, J = 5.6 Hz), 2.50 (3H, s).

HRMS (ESI+) m/z 413.22 (413.44 calculated for C₂₂H₁₅F₃N₂OS+H⁺ [M +H]⁺).

### Compound 9: N-(2-(benzo[d]thiazol-2-yl)-4-methylphenyl)-4-(butylamino)-2,3,5,6-tetrafluorobenzamide

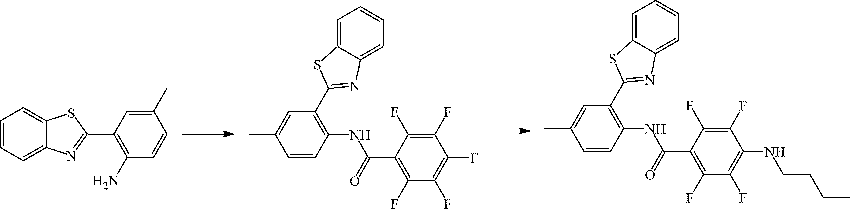 [SMILES: Cc1ccc(N)c(-c2nc3ccccc3s2)c1 ; Cc1ccc(NC(=O)c2c(F)c(F)c(F)c(F)c2F)c(-c2nc3ccccc3s2)c1 ; CCCCNc1c(F)c(F)c(C(=O)Nc2ccc(C)cc2-c2nc3ccccc3s2)c(F)c1F]

Step 1: The same procedure as that described above for the preparation of compound 3 was used with 1.06 g of 2-(1,3-benzothiazol-2-yl)-4-methylaniline, 1.8 mL of pyridine, 9 mL of THF and 0.7 mL of pentafluorobenzoyl chloride to give the intermediate compound (1.51 g).

Step 2: The same procedure as that described above for the preparation of compound 5 was used with 1.55 g of 2-(1,3-benzo[d]thiazol-2-yl)-4-methylaniline, 0.95 g of potassium carbonate, 6 mL of DMF and 0.5 mL of butylamine to give compound 9 (1.33 g).

¹H NMR (CDCl₃): δ 13.10 (1H, s), 8.87 (1H, d, J = 5.6 Hz), 7.93 (1H, d, J = 5.2 Hz), 7.83 (1H, d, J = 5.6 Hz), 7.69 (1H, s), 7.51 (1H, td, J = 5.2, 0.8 Hz), 7.43 (1H, td, J = 5.0, 0.8 Hz), 7.35 (1H, dd, J = 5.6, 0.8 Hz), 4.13 (1H, bs), 3.54 (2H, q, J = 4.4 Hz), 2.44 (3H, s), 1.67 (2H, q, J = 4.8 Hz), 1.47 (2H, h, J = 5.2 Hz), 1.01 (3H, t, J = 4.8 Hz).

HRMS (ESI+) m/z 488.42 (488.52 calculated for C₂₅H₂₁F₄N₃OS+H⁺ [M +H]⁺).

### Compound 10: N-(2-(benzo[d]thiazol-2-yl)-4-methylphenyl)cinnamamide

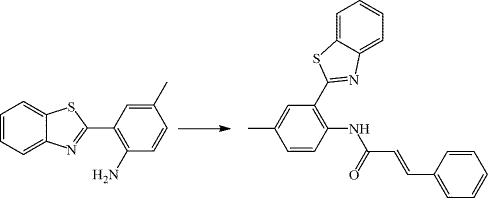 [SMILES: Cc1ccc(N)c(-c2nc3ccccc3s2)c1 ; Cc1ccc(NC(=O)/C=C/c2ccccc2)c(-c2nc3ccccc3s2)c1]

The same procedure as that described above for the preparation of compound 3 was used with 0.5 g of 2-(1,3-benzothiazol-2-yl)-4-methylaniline, 0.8 mL of pyridine, 4 mL of THF and 0.4 mL of cinnamoyl chloride to give compound 10 (0.55 g).

¹H NMR (CDCl₃): δ 12.71 (1H, s), 8.86 (1H, d, J = 5.6 Hz), 8.06 (1H, d, J = 5.6 Hz), 7.95 (1H, d, J = 5.2 Hz), 7.81 (1H, d, J = 10.4 Hz), 7.68 (1H, d, J = 0.8 Hz), 7.67 - 7.64 (2H, m), 7.57 (1H, td, J = 5.2, 0.8 Hz), 7.49 - 7.42 (4H, m), 7.34 (1H, dd, J = 5.6, 1.2 Hz), 6.73 (1H, d, J = 10.4 Hz), 2.43 (3H, s).

HRMS (ESI+) m/z 371.15 (371.47 calculated for C₂₃H₁₈N₂OS+H⁺ [M +H]⁺).

### Compound 11: N-(2-(benzo[d]thiazol-2-yl)-4-methoxyphenyl)benzamide

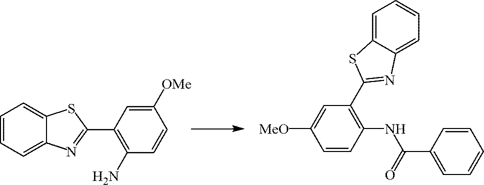 [SMILES: COc1ccc(N)c(-c2nc3ccccc3s2)c1 ; COc1ccc(NC(=O)c2ccccc2)c(-c2nc3ccccc3s2)c1]

The same procedure as that described above for the preparation of compound 3 was used with 1 g of 2-(1,3-benzothiazol-2-yl)-4-methoxyaniline, 1.6 mL of pyridine, 5 mL of THF and 0.4 mL of benzoyl chloride to give compound 11 (1.05 g).

¹H NMR (CDCl₃): δ 13.10 (1H, s), 8.99 (1H, d, J = 6 Hz), 8.23 (2H, dd, J = 5.2, 1.2 Hz), 8.01 (1H, d, J = 5.6 Hz), 7.94 (1H, d, J = 5.6 Hz), 7.63 - 7.59 (3H, m) 7.55 (1H, td, J = 5.2, 0.8 Hz), 7.46 (1H, td, J = 4.8, 0.8 Hz), 7.41 (1H, d, J = 2 Hz), 7.12 (1H, dd, J = 6, 1.2 Hz), 3.92 (3H, s).

HRMS (ESI+) m/z 361.12 (361.44 calculated for C₂₁H₁₆N₂O₂S+H⁺ [M +H]⁺).

### Compound 12: N-(2-(benzo[d]thiazol-2-yl)-4-methoxyphenyl)-4-(trifluoromethyl)benzamide

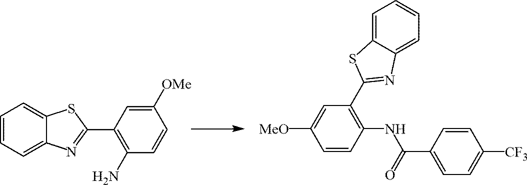 [SMILES: COc1ccc(N)c(-c2nc3ccccc3s2)c1 ; COc1ccc(NC(=O)c2ccc(C(F)(F)F)cc2)c(-c2nc3ccccc3s2)c1]

The same procedure as that described above for the preparation of compound 3 was used with 0.47 g of 2-(1,3-benzothiazol-2-yl)-4-methoxyaniline, 0.6 mL of pyridine, 6 mL of THF and 0.5 mL of trifluoromethylbenzoyl chloride to give compound 12 (0.59 g).

¹H NMR (CDCl₃): δ 13.25 (1H, s), 8.98 (1H, d, J = 6,2 Hz), 8.43 (2H, d, J = 5.6 Hz), 8.34 \\(2H, d, J = 5.6 Hz), 8.14 (1H, d, J = 5.6 Hz), 8.02 (1H, J = 3.8 Hz) 7.98 (1H, d, J = 5.8 Hz), 7.61 (1H, t, J = 4.8 Hz), 7.45 (1H, d, J = 1.6 Hz), 7.15 (1H, dd, J = 6.0, 2.0 Hz), 3.94 (3H, s). HRMS (ESI+) m/z 429.10 (429.43 calculated for C₂₂H₁₅F₃N₂O₂S+H⁺ [M +H]⁺).

### Compound 13: N-(2-(benzo[d]thiazol-2-yl)-4-methoxyphenyl)-4-(butylamino)-2,3,5,6-tetrafluorobenzamide

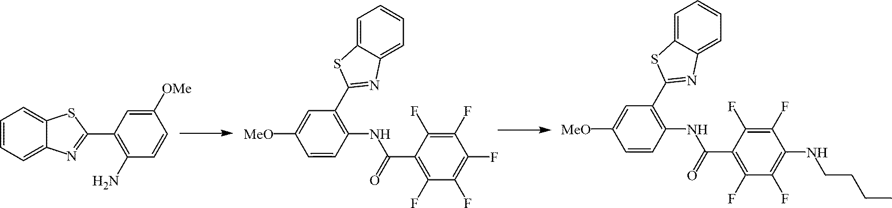 [SMILES: COc1ccc(N)c(-c2nc3ccccc3s2)c1 ; COc1ccc(NC(=O)c2c(F)c(F)c(F)c(F)c2F)c(-c2nc3ccccc3s2)c1 ; CCCCNc1c(F)c(F)c(C(=O)Nc2ccc(OC)cc2-c2nc3ccccc3s2)c(F)c1F]

Step 1: The same procedure as that described above for the preparation of compound 3 was used with 1.32 g of 2-(1,3-benzothiazol-2-yl)-4-methoxyaniline, 2.1 mL of pyridine, 12 mL of THF and 0.8 mL of pentafluorobenzoyl chloride to give the intermediate compound (1.63 g).

Step 2: The same procedure as that described above for the preparation of compound 5 was used with 1.63 g of 2-(1,3-benzo[d]thiazol-2-yl)-4-methoxyaniline, 1.0 g of potassium carbonate, 6 mL of DMF and 0.52 mL of butylamine to give compound 13 (1.43 g).

¹H NMR (CDCl₃): δ 13.30 (1H, s), 8.92 (1H, d, J = 5.8 Hz), 8.14 (1H, d, J = 5.4 Hz), 7.85 (1H, d, J = 5.4 Hz), 7.78 (1H, s), 7.56 (1H, t, J = 4.8 Hz), 7.52 (1H, t, J = 4.8 Hz), 7.19 (1H, d, J = 5.8 Hz), 4.12 (1H, bs), 3.92 (3H, s), 3.54 (2H, q, J = 4.8 Hz), 1.66 (2H, q, J = 4.8 Hz), 1.45 (2H, h, J = 5.2 Hz), 1.02 (3H, t, J = 4.8 Hz).

HRMS (ESI+) m/z 504.17 (504.52 calculated for C₂₅H₂₁F₄N₃O₂S+H⁺ [M +H]⁺).

### Compound 14: N-(2-(benzo[d]thiazol-2-yl)-4-methoxyphenyl)cinnamamide

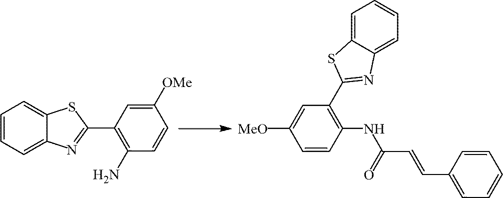 [SMILES: COc1ccc(N)c(-c2nc3ccccc3s2)c1 ; COc1ccc(NC(=O)/C=C/c2ccccc2)c(-c2nc3ccccc3s2)c1]

The same procedure as that described above for the preparation of compound 3 was used with 0.50 g of 2-(1,3-benzothiazol-2-yl)-4-methoxyaniline, 0.77 mL of pyridine, 3 mL of THF and 0.34 mL of trifluoromethylbenzoyl chloride to give compound 14 (0.51 g).

¹H NMR (CDCl₃): δ 12.51 (1H, s), 8.90 (1H, d, J = 6 Hz), 8.08 (1H, d, J = 5.6 Hz), 7.95 (1H, d, J = 5.2 Hz), 7.95 (1H, d, J = 5.2 Hz), 7.80 (1H, d, J = 10.8 Hz), 7.64 (2H, d, J = 4.8 Hz), 7.58 (1H, t, J = 5.2 Hz), 7.49 - 7.46 (3H, m), 7.43 (1H, d, J = 4.8 Hz), 7.39 (1H, d, J = 1.6 Hz), 7.10 (1H, dd, J = 6.0, 1.6 Hz), 6.72 (1H, d, J = 10.4 Hz), 3.91 (3H, s).

HRMS (ESI+) m/z 387.17 (387.47 calculated for C₂₃H₁₅N₂O₂S+H⁺ [M +H]⁺).

### Compound 16: N-(2-(benzo[d]thiazol-2-yl)-4-fluorophenyl)-4-(trifluoromethyl)benzamide

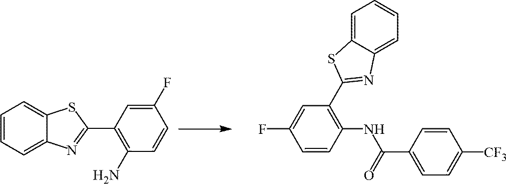 [SMILES: Nc1ccc(F)cc1-c1nc2ccccc2s1 ; O=C(Nc1ccc(F)cc1-c1nc2ccccc2s1)c1ccc(C(F)(F)F)cc1]

The same procedure as that described above for the preparation of compound 4 was used with 0.74 g of 2-(1,3-benzothiazol-2-yl)-4-fluoroaniline, 1.1 mL of pyridine, 3 mL of THF and 0.43 mL of trifluoromethylbenzoyl chloride to give compound 16 (0.78 g).

¹H NMR (CDCl₃): δ 13.37 (1H, s), 9.06 (1H, dd, J = 6, 3.2 Hz), 8.34 (2H, d, J = 5.6 Hz), 7.99 (2H, t, J = 6.2 Hz), 7.88 (2H, d, J = 5.2 Hz), 7.64 - 7.62 (2H, m) 7.52 (1H, t, J = 5.0 Hz), 7.28 (1H, td, J = 5.6, 2.0 Hz).

HRMS (ESI+) m/z 417.13 (417.40 calculated for C₂₁H₁₂F₄N₂OS+H⁺ [M +H]⁺).

### Compound 27: N-(2-(benzo[d]thiazol-2-yl)-5-chlorophenyl)benzamide

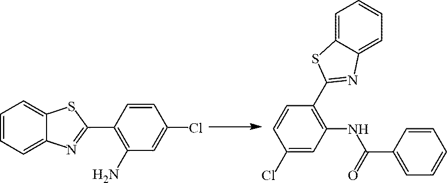 [SMILES: Nc1cc(Cl)ccc1-c1nc2ccccc2s1 ; O=C(Nc1cc(Cl)ccc1-c1nc2ccccc2s1)c1ccccc1]

The same procedure as that described above for the preparation of compound 3 was used with 2 g of 2-(1,3-benzothiazol-2-yl)-5-chloroaniline, 3.1 mL of pyridine, 3 mL of THF and 3.6 mL of benzoyl chloride to give compound 27 (1.36 g).

¹H NMR (CDCl₃): δ 13.48 (1H, s), 9.17 (1H, d, J = 1.2 Hz), 8.24 (2H, dd, J = 5.6 Hz), 8.0 (1H, d, J = 5.2 Hz), 7.95 (1H, d, J = 4.8 Hz), 7.82 (1H, d, J = 5.6 Hz) 7.66 - 7.61 (3H, m), 7.57 (1H, td, J = 5.0, 0.8 Hz), 7.48 (1H, td, J = 5, 0.8 Hz), 7.18 (1H, dd, J = 5.6, 1.2 Hz).

HRMS (ESI+) m/z 365.65 (365.86 calculated for C₂₀H₁₃ClN₂OS+H⁺ [M +H]⁺).

### Compound 29: N-(2-(benzo[d]thiazol-2-yl)-5-chlorophenyl)-4-(butylamino)-2,3,5,6-tetrafluorobenzamide

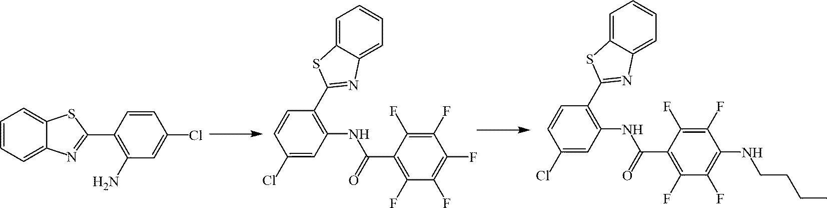 [SMILES: Nc1cc(Cl)ccc1-c1nc2ccccc2s1 ; O=C(Nc1cc(Cl)ccc1-c1nc2ccccc2s1)c1c(F)c(F)c(F)c(F)c1F ; CCCCNc1c(F)c(F)c(C(=O)Nc2cc(Cl)ccc2-c2nc3ccccc3s2)c(F)c1F]

Step 1: The same procedure as that described above for the preparation of compound 3 was used with 2.00 g of 2-(1,3-benzothiazol-2-yl)-5-chloroaniline, 3.1 mL of pyridine, 10 mL of THF and 3.3 mL of pentafluorobenzoyl chloride to give the intermediate compound (1.45 g).

Step 2: The same procedure as that described above for the preparation of compound 5 was used with 1.45 g of 2-(1,3-benzo[d]thiazol-2-yl)-5-chloroaniline, 0.88 g of potassium carbonate, 5 mL of DMF and 0.46 mL of butylamine to give compound 29 (0.93 g).

¹H NMR (CDCl₃): δ 13.31 (1H, s), 9.09 (1H, d, J = 1.2 Hz), 7.93 (1H, d, J =5.6 Hz), 7.84 (1H, d, J = 5.2 Hz), 7.80 (1H, d, J = 5.2 Hz), 7.52 (1H, td, J = 5.2, 0.8 Hz), 7.45 (1H, td, J = 5.0, 0.8 Hz), 7.2 (1H, dd, J = 5.6, 1.2 Hz), 3.55 (2H, q, J = 4.8 Hz), 1.68 (2H, q, J = 5.2 Hz), 1.48 (2H, h, J = 5.2 Hz), 1.02 (3H, t, J = 4.8 Hz).

HRMS (ESI+) m/z 508.63 (508.94 calculated for C₂₄H₁₈C1F₄N₃OS+H⁺ [M +H]⁺).

### Compound 35: N-(2-(benzo[d]thiazol-2-yl)-5-chlorophenyl)-2,3,5,6-tetrafluoro-4-((2-hydroxyethyl)amino)benzamide

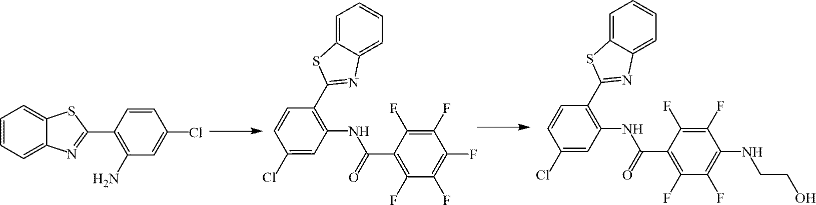 [SMILES: Nc1cc(Cl)ccc1-c1nc2ccccc2s1 ; O=C(Nc1cc(Cl)ccc1-c1nc2ccccc2s1)c1c(F)c(F)c(F)c(F)c1F ; O=C(Nc1cc(Cl)ccc1-c1nc2ccccc2s1)c1c(F)c(F)c(NCCO)c(F)c1F]

Step 1: The same procedure as that described above for the preparation of compound 3 was used with 2.00 g of 2-(1,3-benzothiazol-2-yl)-5-chloroaniline, 3.1 mL of pyridine, 10 mL of THF and 3.3 mL of pentafluorobenzoyl chloride to give the intermediate compound (1.45 g).

Step 2: The same procedure as that described above for the preparation of compound 4 was used with 1.0 g of 2-(1,3-benzo[d]thiazol-2-yl)-5-chloroaniline, 0.66 g of potassium carbonate, 5 mL of DMF and 0.43 mL of aminoethanol to give compound 35 (0.75 g).

¹H NMR (CDCl₃): δ 13.34 (1H, s), 9.08 (1H, d, J = 1.2 Hz), 7.92 (1H, d, J =5.2 Hz), 7.83 (1H, d, J = 5.6 Hz), 7.80 (1H, d, J = 6.0 Hz), 7.52 (1H, td, J = 5.2, 0.8 Hz), 7.44 (1H, td, J = 5.2, 0.8 Hz), 7.2 (1H, dd, J = 5.6, 1.2 Hz), 4.7 (1H, bs), 3.94 (2H, t, J = 3.2 Hz), 3.73 (2H, m).

HRMS (ESI+) m/z 496.57 (496.88 calculated for C₂₂H₁₄ClF₄N₃OS+H⁺ [M +H]⁺).

### APPLICATIONS

### Example 1: Preparation of a Fluorescent Composition in Layer Form and Use Thereof for Securement

For this example, the compound having formula I used is compound 13 having the formula hereinafter:

### [Chem. 10]

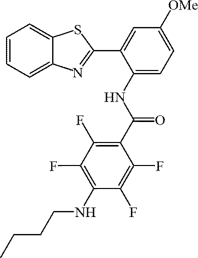 [SMILES: CCCCNc1c(F)c(F)c(C(=O)Nc2ccc(OC)cc2-c2nc3ccccc3s2)c(F)c1F]

Compound 13 is in the form of a yellow powder and absorbs at 365 nm and has a fluorescence emission at 605 nm. 17 grams of compound are mixed with 10 kg of polycarbonate (PC Makralon 2456) using an extruder so as to obtain the fluorescent composition.

The extrusion is carried out on a twin-screw extruder (Brabender) with a screw rotation speed of 50 rom, a hopper flow rate of 3.8 kg/h and the following temperature profile: 275° C. -280° C. -280° C. -285° C. -285° C. -285° C. -290° C. (feed => line).

From the extruded fluorescent composition, a quantity is removed and then diluted 10 times by adding polycarbonate (PC Makralon 2456) through a Fairex extruder (diameter 45) equipped with Scamex flat dies of a width of 350 mm, followed by a 3-cylinder roller having an inclination of 30°.

At the calender output, the fluorescent composition i s then in the form of a layer of thickness of 100 mm. After setting the calender, the test was reproduced so as to obtain a second layer of a thickness of 400 mm from the fluorescent composition.

The secured layers thus obtained are then used as a document substrate, said document thus being secured.

The visual and spectrophotometric analyses of the layers obtained show that the incorporation of the fluorescent dye in the polycarbonate does not alter the performances thereof in terms of absorption and fluorescence emission. The fluorescent layers obtained show similar properties to those observed when the compound is in solution.

### Example 2: Preparation of a Fluorescent Composition in Varnish Form and Use Thereof for Securement.

For this example, the compound having formula I used is compound 14 having the formula hereinafter:

### [Chem. 11]

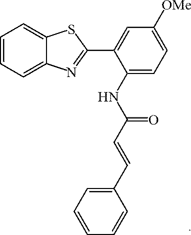

44 mg of compound 14 is integrated in 10 g of solvent-based acrylic varnish (0.4% weight/weight).

The fluorescent varnish thus obtained is then coated on a polyethylene terephthalate film of 100 µm in thickness using an automatic applicator (automatic film applicator, TQC ASTM D 823) and a 20 µm Meyer rod with an application rate of 50 mm/s.

The measured varnish deposition has a thickness of 5 µm in keeping with a solvent-based varnish having 33% dry extract.

The assembly consisting of the polyethylene terephthalate film coated with fluorescent varnish forms a fluorescent layer. The fluorescent layer thus obtained is a secured layer. When it is illuminated with a UV lamp (365 nm), a purple fluorescence appears to the naked eye.

A visual and spectrophotometric analysis of the secured layer shows that the incorporation of the fluorescent dye in the polycarbonate does not alter the performances thereof in terms of absorption and fluorescence emission.

The secured layer thus obtained, by means of the fluorescence properties thereof, is used as a card substrate.

### Example 3: Preparation of a Fluorescent Composition in Ink Form Comprising A Compound Having Formula I According to the Invention and a Compound Having Formula III

For this example, the compound having formula I used is compound 27 having the formula hereinafter:

### [Chem. 12]

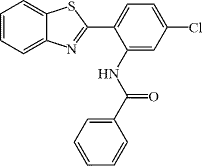 [SMILES: O=C(Nc1cc(Cl)ccc1-c1nc2ccccc2s1)c1ccccc1]

Compound 27 is in the form of a white powder, absorbs at 365 nm, has a fluorescence emission at 522 nm.

The compound having formula III used is compound 36 having the formula hereinafter:

### [Chem. 13]

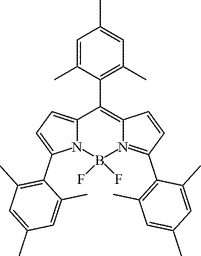

Compound 36 is in the form of an orange powder, absorbs at 547 nm, has a fluorescence emission at 568 nm.

Preparation of the fluorescent composition in ink form:

400 mg of compound 27 is uniformly dissolved in 100 g of Mara® Gloss GO type transparent unexposed screen-printing ink distributed by Marabu compatible with printing on a polycarbonate plastic surface.

100 mg of compound 36 is uniformly dissolved in 100 g of the same transparent unexposed screen-printing ink.

The ink containing compound 27 and the ink containing compound 36 are then mixed according to a weight ratio of 1:1.

The fluorescent composition thus formulated is printed on a transparent polycarbonate card.

Results: The fluorescent composition printed perfectly.

The pattern thus printed has a pink colour visible by transparency when said card is disposed on a white background, and an orange colour visible by transparency when said card is disposed on a black background.

When the card is illuminated under UV, a yellow fluorescence is visible.

### Example 4: Preparation of a Fluorescent Composition in Ink Form Comprising A Compound Having Formula I According to the Invention

For this example, the compound of family I used is compound 3 having the formula hereinafter:

### [Chem. 14]

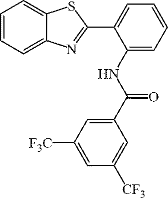 [SMILES: O=C(Nc1ccccc1-c1nc2ccccc2s1)c1cc(C(F)(F)F)cc(C(F)(F)F)c1]

Compound 3 is in the form of a white powder, absorbs at 365 nm, has a fluorescence emission at 513 nm.

Preparation of the fluorescent composition in ink form: 400 mg of compound 3 is uniformly dissolved in 100 g of Mara® Gloss GO type transparent unexposed screen-printing ink distributed by Marabu compatible with printing on a polycarbonate plastic surface. The mass concentration of the ink is therefore 0.4% (w/w). The fluorescent composition thus formulated is printed on a transparent polycarbonate card using a mesh size 90 screen-printing frame.

### Results: The Fluorescent Composition Printed Perfectly

The pattern thus printed is invisible under ambient lighting and has an intense yellow fluorescence under UV lighting (365 nm).

The optical properties of the dye are transposed between the pure dye and the screen-printed layer.

### Example 5: Preparation of a Fluorescent Composition in Ink Form Comprising A Compound Having Formula I According to the Invention

For this example, the compound having formula I used is compound 13 having the formula hereinafter:

### [Chem. 10]

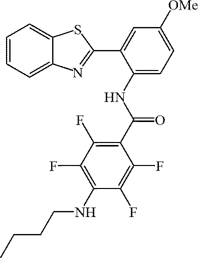 [SMILES: CCCCNc1c(F)c(F)c(C(=O)Nc2ccc(OC)cc2-c2nc3ccccc3s2)c(F)c1F]

Compound 13 is in the form of a white powder, absorbs at 365 nm, has a fluorescence emission at 605 nm.

Preparation of the fluorescent composition in ink form: 400 mg of compound 3 is uniformly dissolved in 100 g of Mara® Gloss GO type transparent unexposed screen-printing ink distributed by Marabu compatible with printing on a polycarbonate plastic surface. The mass concentration of the ink is therefore 0.4% (w/w). The fluorescent composition thus formulated is printed on a transparent polycarbonate card using a mesh size 90 screen-printing frame.

### Results: The Fluorescent Composition Printed Perfectly

The pattern thus printed is invisible under ambient lighting and has a red fluorescence under UV lighting (365 nm).

The optical properties of the dye are transposed between the pure dye and the screen-printed layer.

### Example 6: Preparation of a Fluorescent Composition in Ink Form Comprising Two Compounds Having Formula I According to the Invention

For this example, the compound of family I used are compounds 3 and 13 the formulas of which are presented hereinafter:

### [Chem. 14]

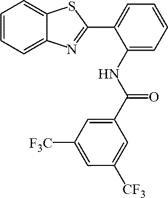 [SMILES: O=C(Nc1ccccc1-c1nc2ccccc2s1)c1cc(C(F)(F)F)cc(C(F)(F)F)c1]

### [Chem. 10]

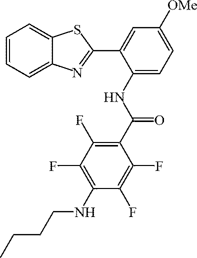 [SMILES: CCCCNc1c(F)c(F)c(C(=O)Nc2ccc(OC)cc2-c2nc3ccccc3s2)c(F)c1F]

Preparation of the fluorescent composition in ink form:

400 mg of compound 3 is uniformly dissolved in 100 g of Mara® Gloss GO type transparent unexposed screen-printing ink distributed by Marabu compatible with printing on a polycarbonate plastic surface.

400 mg of compound 13 is uniformly dissolved in 100 g of the same transparent unexposed screen-printing ink.

The two inks are mixed with a mass ratio of 3:1 (compound 13 : compound 3 - by weight) and homogenised until a uniform colour is obtained.

Results: The fluorescent composition printed perfectly.

The pattern thus printed is invisible under ambient lighting and has an intense orange fluorescence under UV lighting (365 nm).

The colour thus obtained refers to the mixtures of the respective fluorescence colours of the two dyes.

1-19. (canceled)

20. A fluorescent composition comprising a polymer matrix incorporating a compound having formula I: 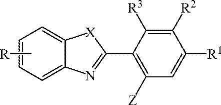
wherein,
X is chosen from NH, O and S;
Z is chosen from OH, NHR⁵ and N(R⁵)₂;
R is chosen from hydrogen, C1-C6-alkyl, C3-C6-cycloalkyl, aryl, vinyl, ethynyl, halogen, —NO₂, —NH₂, —NHR⁴, —N(R⁴)₂, —N⁺(R⁴)₃, —NHCOR⁴, —CHO, —C(O)OH, —C(O)OR⁴, —CF₃, C1-C6-alkoxy, aryloxy, —SH, —SO₃H, —SR⁴;
R¹, R² and R³ are independently chosen from hydrogen, C1-C6-alkyl, C3-C6-cycloalkyl, aryl, vinyl, ethynyl, halogen, —NO₂, —NH₂, —NHR⁴, —N(R⁴)₂, —N⁺(R⁴)₃, —NHCOR⁴, —CHO, —C(O)OH, —C(O)OR⁴, —CF₃, C1-C6-alkoxy, aryloxy, —SH, —SO₃H, —SR⁴; R⁴ is chosen from C1-C6-alkyl, C3-C6-cycloalkyl and aryl; R⁵ is chosen from C1-C6-alkyl, hydroxy-C1-C6-alkyl, halogen, aryl, acyl, C1-C6-alkyloxycarbonyl, C1-C6-alkylaminocarbonyl, C1-C6-alkylaminothionyl, di-C1-C6-alkylaminothionyl, arylaminocarbonyl, arylaminothionyl, arylsulfonyl, C1-C6-alkylsulfonyl, cinnamoyl, benzoyl, 2,3,4,5,6-pentahalogenobenzoyl and 2,3,5,6-tetrahalogenobenzoyl, optionally substituted by C1-C6-alkyl, C1-C6-alkoxy, trifluoro-C1-C6-alkyl, C1-C6-alkylamino or hydroxy-C1-C6-alkylamino;with the condition that Z is not OH when R² is —NHCOR⁴; and
wherein the symbol 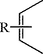
means that R can be substituted several times at any free position of the benzene nucleus.

21. The fluorescent composition according to claim 20, wherein the polymer of the matrix is chosen from polycarbonate, polystyrene, polyethylene, polypropylene, polyethylene terephthalate, polyacrylate, polymethacrylate, polyvinyl chloride, polyamides, polyaramids, ethylene-vinyl acetate, polyurethane, thermoplastic polyurethane (TPU), cyanoacrylate, rosin resins, pine resins, photopolymerisable resins or mixtures thereof.

22. The fluorescent composition according to claim 20, wherein X is S in the compound having formula I.

23. The fluorescent composition according to claim 20, wherein Z is NHR⁵ or N(R⁵)₂ in the compound having formula I.

24. The fluorescent composition according to claim 20, wherein R is hydrogen in the compound having formula I.

25. The fluorescent composition according to claim 20, wherein the compound having formula I is chosen from:
N-(2-(benzo[d]thiazol-2-yl)phenyl)benzamide;
N-(2-(benzo[d]thiazol-2-yl)phenyl)-4-(trifluoromethyl)benzamide;
N-(2-(benzo[d]thiazol-2-yl)phenyl)-3,5-bis(trifluoromethyl)benzamide;
N-(2-(benzo[d]thiazol-2-yl)phenyl)-2,3,4,5,6-pentafluorobenzamide;
N-(2-(benzo[d]thiazol-2-yl)phenyl)-4-(butylamino)-2,3,5,6-tetrafluorobenzamide;
N-(2-(benzo[d]thiazol-2-yl)phenyl)cinnamamide;
N-(2-(benzo[d]thiazol-2-yl)-4-methylphenyl)benzamide
N-(2-(benzo[d]thiazol-2-yl)-4-methylphenyl)-4-(trifluoromethyl)benzamide;
N-(2-(benzo[d]thiazol-2-yl)-4-methylphenyl)-4-(butylamino)-2,3,5,6-tetrafluorobenzamide;
N-(2-(benzo[d]thiazol-2-yl)-4-methylphenyl)cinnamamide;
N-(2-(benzo[d]thiazol-2-yl)-4-methoxyphenyl)benzamide;
N-(2-(benzo[d]thiazol-2-yl)-4-methoxyphenyl)-4-(trifluoromethyl)benzamide;
N-(2-(benzo[d]thiazol-2-yl)-4-methoxyphenyl)-4-(butylamino)-2,3,5,6-tetrafluorobenzamide;
N-(2-(benzo[d]thiazol-2-yl)-4-methoxyphenyl)cinnamamide;
N-(2-(benzo[d]thiazol-2-yl)-4-fluorophenyl)benzamide;
N-(2-(benzo[d]thiazol-2-yl)-4-fluorophenyl)-4-(trifluoromethyl)benzamide;
N-(2-(benzo[d]thiazol-2-yl)-4-fluorophenyl)-4-(butylamino)-2,3,5,6-tetrafluorobenzamide;
N-(2-(benzo[d]thiazol-2-yl)-4-fluorophenyl)cinnamamide;
N-(2-(benzo[d]thiazol-2-yl)-4-nitrophenyl)benzamide;
N-(2-(benzo[d]thiazol-2-yl)-4-nitrophenyl)-4-(trifluoromethyl)benzamide;
N-(2-(benzo[d]thiazol-2-yl)-4-nitrophenyl)-4-(butylamino)-2,3,5,6-tetrafluorobenzamide;
N-(2-(benzo[d]thiazol-2-yl)-4-nitrophenyl)cinnamamide;
N-(2-(benzo[d]thiazol-2-yl)-5-methoxyphenyl)benzamide;
N-(2-(benzo[d]thiazol-2-yl)-5-methoxyphenyl)-4-(trifluoromethyl)benzamide;
N-(2-(benzo[d]thiazol-2-yl)-5-methoxyphenyl)-4-(butylamino)-2,3,5,6-tetrafluorobenzamide;
N-(2-(benzo[d]thiazol-2-yl)-5-methoxyphenyl)cinnamamide;
N-(2-(benzo[d]thiazol-2-yl)-5-chlorophenyl)benzamide;
N-(2-(benzo[d]thiazol-2-yl)-5-chlorophenyl)-4-(trifluoromethyl)benzamide;
N-(2-(benzo[d]thiazol-2-yl)-5-chlorophenyl)-4-(butylamino)-2,3,5,6-tetrafluorobenzamide;
N-(2-(benzo[d]thiazol-2-yl)-5-chlorophenyl)cinnamamide;
N-(2-(benzo[d]thiazol-2-yl)-3-chlorophenyl)benzamide;
N-(2-(benzo[d]thiazol-2-yl)-3-chlorophenyl)-4-(trifluoromethyl)benzamide;
N-(2-(benzo[d]thiazol-2-yl)-3-chlorophenyl)-4-(butylamino)-2,3,5,6-tetrafluorobenzamide;
N-(2-(benzo[d]thiazol-2-yl)-3-chlorophenyl)cinnamamide; and
N-(2-(benzo[d]thiazol-2-yl)-5-chlorophenyl)-2,3,5,6-tetrafluoro-4-((2-hydroxyethyl)amino)benzamide.

26. The fluorescent composition according to claim 20, wherein the fluorescent composition further comprises a second compound having formula I.

27. The fluorescent composition according to claim 20, wherein the fluorescent composition further comprises a second compound having formula III: 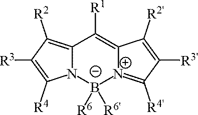
wherein,
R¹ is C1 to C6 alkyl, C5 to C6 cycloalkyl, C5 to C6 heteroalkyl, phenyl, said phenyl group being optionally substituted by one or more groups chosen from C1 to C2 alkyl, hydroxy, R⁵COO⁻ and halogen;
R² and R²′ are independently chosen from hydrogen and C1 to C2 alkyl;
R³ and R³′ are independently chosen from hydrogen, aryl, heteroaryl, cycloalkyl, alkyl, alkenyl, alkynyl, said aryl, heteroaryl, cycloalkyl, alkyl, alkenyl and alkynyl being optionally substituted by one or more groups chosen from C1 to C4 alkyl, aryl, hydroxy and ferrocene, said aryl group being optionally substituted by one or more groups chosen from aryl, C1 to C2 alkyl, halogen, hydroxy, dimethylamino, nitro, said aryl being optionally substituted by a C1 to C2 alkyl group;
R⁴ and R^{4′} are independently chosen from aryl, heteroaryl, cycloalkyl, alkyl, alkenyl, said aryl, heteroaryl, cycloalkyl, alkyl and alkenyl being optionally substituted by one or more groups chosen from C1 to C3 alkyl, aryl, hydroxy and ferrocene, said aryl group being optionally substituted by one or more groups chosen from aryl, C1 to C2 alkyl, halogen, hydroxy, dimethylamino, nitro, said aryl being optionally substituted by a C1 to C2 alkyl group;
R⁵ is C1 to C4 alkyl or C2 to C4 alkenyl.
R⁶ and R⁶′ are independently chosen from halogens, C1 to C4 alkoxy, C2 to C4 alkenoxy, C1 to C4 alkyl, C2 to C4 alkenyl, CN or aryl, said aryl being optionally substituted by one or more groups chosen from C1 to C2 alkyl, hydroxy, R⁵COO— and halogen.

28. The fluorescent composition according to claim 20, wherein the fluorescent composition is adapted for securement of a product.

29. The fluorescent composition according to claim 28, wherein the product to be secured is a document.

30. The fluorescent composition according to claim 28, wherein the fluorescent composition is a fluorescent ink adapted for printing.

31. The fluorescent composition according to claim 30, wherein the printing is chosen from screen printing, offset printing, flexography, heliography, ink-jet printing, digital printing, copperplate printing and 3D printing.

32. A method for the securement of a product comprising a step of preparing a fluorescent composition as defined according to claim 20 and a securement step by applying said fluorescent composition on at least a portion of the product to be secured.

33. The method for the securement of a product according to claim 32, wherein the securement step is carried out by rolling, printing, weaving, bonding, coating or impregnation.

34. The method for the securement of a product according to claim 32, wherein the method comprises a step of shaping the fluorescent composition before the securement step.

35. The method for the securement of a product according to claim 32, wherein the fluorescent composition is a fluorescent ink and in that the securement step is carried out by printing.

36. The method for the securement of a product according to claim 32, wherein the product is an identity, fiduciary or administrative document.

37. A compound having formula (II): 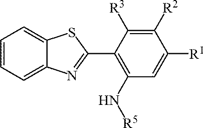
wherein,
R¹, and R² and R³ are independently chosen from hydrogen, C1-C6-alkyl, C3-C6-cycloalkyl, aryl, vinyl, ethynyl, halogen, —NO₂, —NH₂, —NHR⁴, —N(R⁴)₂, —N⁺(R⁴)₃, —NHCOR⁴, —CHO, —C(O)OH, —C(O)OR⁴, —CF₃, C1-C6-alkoxy, aryloxy, —SH, —SO₃H, —SR⁴;
R⁴ is chosen from C1-C6-alkyl, C1-C6-cycloalkyl and aryl;
R⁵ is chosen from C1-C6-alkyl, hydroxy-C1-C6-alkyl, C1-C6-alkyloxycarbonyl, di-C1-C6-alkylaminothionyl, C1-C6-alkylsulfonyl, cinnamoyl, benzoyl, 2,3,4,5,6-pentahalogenobenzoyl and 2,3,5,6-tetrahalogenobenzoyl, optionally substituted by alkyl, C1-C6-alkoxy, trifluoro-Cl-C6-alkyl, C1-C6-alkylamino or hydroxy-C1-C6-alkylamino; and
with the condition that:
R¹, R² and R³ are not all three hydrogen when R⁵ is cinnamoyl, or benzoyl optionally substituted by methyl, methoxy, chloro or trifluoromethyl;
R¹ is not methyl when R⁵ is benzoyl;
R² is not methyl when R⁵ is benzoyl; and
R³ is not methyl, methoxy or chloro when R⁵ is benzoyl.

38. The compound according to claim 37, chosen from:
N-(2-(benzo[d]thiazol-2-yl)phenyl)-3,5-bis(trifluoromethyl)benzamide;
N-(2-(benzo[d]thiazol-2-yl)phenyl)-2,3,4,5,6-pentafluorobenzamide;
N-(2-(benzo[d]thiazol-2-yl)phenyl)-4-(butylamino)-2,3,5,6-tetrafluorobenzamide;
N-(2-(benzo[d]thiazol-2-yl)-4-methylphenyl)-4-(trifluoromethyl)benzamide;
N-(2-(benzo[d]thiazol-2-yl)-4-methylphenyl)-4-(butylamino)-2,3,5,6-tetrafluorobenzamide;
N-(2-(benzo[d]thiazol-2-yl)-4-methylphenyl)cinnamamide;
N-(2-(benzo[d]thiazol-2-yl)-4-methoxyphenyl)benzamide;
N-(2-(benzo[d]thiazol-2-yl)-4-methoxyphenyl)-4-(trifluoromethyl)benzamide;
N-(2-(benzo[d]thiazol-2-yl)-4-methoxyphenyl)-4-(butylamino)-2,3,5,6-tetrafluorobenzamide;
N-(2-(benzo[d]thiazol-2-yl)-4-methoxyphenyl)cinnamamide;
N-(2-(benzo[d]thiazol-2-yl)-4-fluorophenyl)-4-(trifluoromethyl)benzamide;
N-(2-(benzo[d]thiazol-2-yl)-5-chlorophenyl)benzamide;
N-(2-(benzo[d]thiazol-2-yl)-5-chlorophenyl)-4-(butylamino)-2,3,5,6-tetrafluorobenzamide; and
N-(2-(benzo[d]thiazol-2-yl)-5-chlorophenyl)-2,3,5,6-tetrafluoro-4-((2-hydroxyethyl)amino)benzamide.
